(19) United States
(12) Patent Application Publication
(10) Pub. No.: US 2023/0181057 A1
(43) Pub. Date: Jun. 15, 2023

(54) Systems and Methods of Longitudinal Analysis of Human Running Gait Metrics

(71) Applicant: THE REGENTS OF THE UNIVERSITY OF MICHIGAN, Ann Arbor, MI (US)

(72) Inventor: Geoffrey T. Burns, Ann Arbor, MI (US)

(21) Appl. No.: 18/079,828

(22) Filed: Dec. 12, 2022

Related U.S. Application Data
(60) Provisional application No. 63/289,105, filed on Dec. 13, 2021.

(51) Int. Cl. A61B 5/11; A63B 24/00

(52) U.S. Cl. CPC A61B 5/1118; A63B 24/0062; A61B 5/0205

(57) ABSTRACT

Systems and methods relate to personalized analysis of physiology and/or biomechanical behaviors of a runner across one, two, three, four or more running sessions. Particularly, the personalized analysis may examine the relationships between variables of interest (VOIs) and their predictors, both within a single running session and across multiple running sessions. These relationship modeling techniques may be used, for example to gain insights into running performance, injury, training adaptation, and/or external effect attribution.

### CROSS-REFERENCE TO RELATED APPLICATIONS

This application claims priority to and the benefit of the filing date of U.S. Provisional Patent Application No. 63/289,105, filed Dec. 13, 2021 and entitled “Systems and Methods of Longitudinal Analysis of Human Running Metrics,” the entirety of the disclosure of which is hereby incorporated by reference herein.

### FIELD OF DISCLOSURE

The present disclosure is generally directed to the field of human physiology and biomechanics, and more particularly, to sensor arrangements and analytical frameworks for evaluating human running gait and other performance metrics over time.

### BACKGROUND

Research in the field of biomechanics has devoted extensive study to the biomechanical and physiological behavior of the human body while running, including the varying movements of the human body parts to support running, the mechanical interaction of these body parts with the ground, and the metabolic processes the body uses to support the expenditure of energy during and after running. Generally speaking, goals pursuing these studies have included identifying the fundamental biomechanical relationships that express the running gait of human runners, anticipating and preventing acute or stress-induced injuries to runners, and identifying improvements to gait or technique that might improve runners' performance.

Attempts have been made to develop mathematical models that use a limited number of variables to robustly model the human running behavior, and more particularly, to model the elastic behavior of the body as it arrives in and exits contact with the ground with each step. Research has arrived at a “spring-loaded inverted pendulum” model (SLIP, also sometimes referred to as a “spring-mass model”), which, for a given runner having a given mass and forward velocity, characterizes the runner's gait via four primary parameters including leg stiffness, touchdown angle of the leg with the running surface (simply “ground”), leg length, and contact time with the ground.

The SLIP model (or “template”) has, as a basic framework, proven both descriptive and predictive of other biomechanical running gait metrics (e.g., stance time, aerial time, step (stride) frequency, center of motion movement, ground reaction force (GRF), etc.). Still, though, augmentations to the SLIP model have been proposed, e.g., to introduce additional parameters to account for differences between SLIP model predictions and observed running gait metrics. Additionally, where some approaches to obtaining SLIP parameters for a runner rely upon discrete observations during the gait cycle (e.g. maximal vertical force) and simplifying assumptions (e.g. leg length), alternative approaches have been proposed. One approach, developed by Geoffrey T. Burns, Richard Gonzalez, and Ronald F. Zernicke, models runners as spring-mass systems by determining the four primary SLIP parameters using nonlinear regression (NLR) and a full observed vertical ground reaction force (vGRF) time series without additional inputs and fewer traditional parameter assumptions (see Burns, G. T., Gonzalez, R., & Zernicke, R. F. (2021). Improving spring-mass parameter estimation in running using nonlinear regression methods. The Journal of experimental biology, 224 (Pt 6), jeb232850. https://doi.org/10.1242/jeb.232850, the entirety of which is hereby incorporated by reference). This approach derives and validates a time-dependent vGRF function characterized by the four spring-mass parameters of stiffness, touchdown angle, leg length and contact time using a sinusoidal approximation. Comparison of the NLR-estimated spring-mass parameters with traditional calculations in runners using independent and mixed-effects models demonstrated that the mixed-effect NLR method modeled the spring-mass parameters best compared to a conventional sinusoid approximation, the ME NLR estimations by comparison providing similar stiffness approximations, moderately steeper touchdown angles, longer effective leg lengths, and shorter effective contact times. Together, these vGRF-driven system parameters more closely approximated the observed vertical impulses than traditional methods.

Accordingly, researchers have employed the SLIP model and augmentations thereof to attempt to understand biomechanical behavior of runners, whether those runners are novices, amateurs, or athletes with elite-level training. Biomechanical behaviors of runners at different skill levels have been analyzed with respect to the SLIP model, identifying and comparing SLIP parameters of the runners at different skill levels to attempt to identify individualized optimal running biomechanics. See Burns, G. T., Gonzalez, R., Zendler, J. M. & Zernicke R. F. (2021). Bouncing behavior of sub-four minute milers. Scientific Reports 11(10501). https://doi.org/10.1038/s41598-021-89858-1 Moreover, changes in biomechanical behaviors within a same runner have been analyzed over the course of a single race (e.g., step frequency during a 100-km ultramarathon) to attempt to identify how these changes may be associated with fatigue or injury. See Burns, G. T., Zendler, J. M., & Zernicke, R. F. (2019). Step frequency patterns of elite ultramarathon runners during a 100-km road race. Journal of applied physiology (Bethesda, Md.: 1985), 126(2), 462-468. https://doi.org/10.1152/japplphysio1.00374.2018.

However, existing frameworks have not fully modeled variations in biomechanical or physiological relationships within a same runner over greater periods of time (e.g., a series of races or running sessions), nor considered the implications of these variations toward health, performance, and the greater field of biomechanics and exercise physiology.

### SUMMARY

Systems and methods described herein provide for personalized analysis of biomechanical behaviors and/or physiological characteristics of a single runner across one, two, three, four or more running sessions. Particularly, the personalized analysis may examine the relationships between variables of interest (VOIs, e.g., biomechanical and other physiological metrics) and their predictors, both within a single running session and across multiple running sessions. By modeling the relationships of VOIs to predictors, especially continuous predictors, across time both within and across sessions with data selectively managed from ecological collections (e.g., outside the lab in daily living), insights into performance, injury, training adaptation, and external effect attribution can be gained above and beyond traditional approaches or discrete analyses.

In embodiments, a computer-implemented method is provided. The method may include, via one or more processors, (1) obtaining, via one or more sensing devices, for each of a plurality of running sessions of a human runner, sensor data indicative of the respective running session, (2) for each respective running session, determining, based upon the sensor data, values of a variable of interest (VOI) and a predictor variable for a respective plurality of data points over the respective running session, (3) for each respective running session, modeling a statistical relationship between at least the VOI and the predictor variable, and/or (4) based upon the modeled relationship for each respective running session, identifying a change in the statistical relationship between the VOI and predictor variable over the plurality of running sessions. The method may include additional, fewer, and/or alternate actions, including actions described in this detailed description.

In other embodiments, a computing system is provided. The computing system may include one or more processors and one or more computer memories storing non-transitory, computer executable instructions that, when executed via the one or more processors, cause the computing system to (1) (1) obtain, via one or more sensing devices, for each of a plurality of running sessions of a human runner, sensor data indicative of the respective running session, (2) for each respective running session, determine, based upon the sensor data, values of a variable of interest (VOI) and a predictor variable for a respective plurality of data points over the respective running session, (3) for each respective running session, model a statistical relationship between at least the VOI and the predictor variable, and/or (4) based upon the modeled relationship for each respective running session, identify a change in the statistical relationship between the VOI and predictor variable over the plurality of running sessions. The computing system may be configured to perform additional, fewer, and/or alternate actions, in various embodiments.

In still other embodiments, one or more computer readable media are provided. The one or more computer readable media store non-transitory, computer executable instructions that, when executed via one or more processors of one or more computers, cause the one or more computers to (1) obtain, via one or more sensing devices, for each of a plurality of running sessions of a human runner, sensor data indicative of the respective running session, (2) for each respective running session, determine, based upon the sensor data, values of a variable of interest (VOI) and a predictor variable for a respective plurality of data points over the respective running session, (3) for each respective running session, model a statistical relationship between at least the VOI and the predictor variable, and/or (4) based upon the modeled relationship for each respective running session, identify a change in the statistical relationship between the VOI and predictor variable over the plurality of running sessions. The one or more computer readable media may store additional, fewer, and/or alternate instructions, in various embodiments.

### BRIEF DESCRIPTION OF DRAWINGS

The figures described below depict various aspects of the systems and methods disclosed herein. Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive. Further, wherever possible, the following description refers to the reference numerals included in the following figures, in which features depicted in multiple figures are designated with consistent reference numerals.

FIG. 1 illustrates an example sensor arrangement configured to obtain various biomechanical and/or physiological metrics of a human runner;

FIG. 2A illustrates the spring-loaded inverted pendulum (SLIP) model of gait analysis;

FIG. 2B illustrates the SLIP model applied to a human runner;

FIG. 3 illustrates a curve of a ground reaction force (GRF) associated with a step by a runner;

FIG. 4 illustrates changes in vertical and horizontal kinetic energy and elastic and gravitational potential energy through a step cycle;

FIG. 5A illustrates a plot of a variable of interest (VOI) to a predictor over a first running session;

FIG. 5B illustrates a plot of the VOI to the predictor over a second running session;

FIG. 5C illustrates a plot of the VOI to the predictor over a third running session;

FIG. 5D illustrates a plot of the VOI to the predictor over a fourth running session;

FIG. 5E illustrates a plot of the VOI to the predictor over a fifth running session;

FIG. 6A illustrates a plot of a first modeled coefficient relating the VOI to the predictor across a plurality of running sessions;

FIG. 6B illustrates a plot of a second modeled coefficient relating the VOI to the predictor across the plurality of running sessions;

FIG. 6C illustrates a plot of a third modeled coefficient relating the VOI to the predictor across the plurality of running sessions; and

FIG. 7 illustrates a block diagram of an example computer-implemented method, in accordance with at least some of the techniques described herein.

### DETAILED DESCRIPTION

The present disclosure generally relates to the field of human physiology and biomechanics, and more particularly, to sensor arrangements and analytical tools for evaluating human running gait over time.

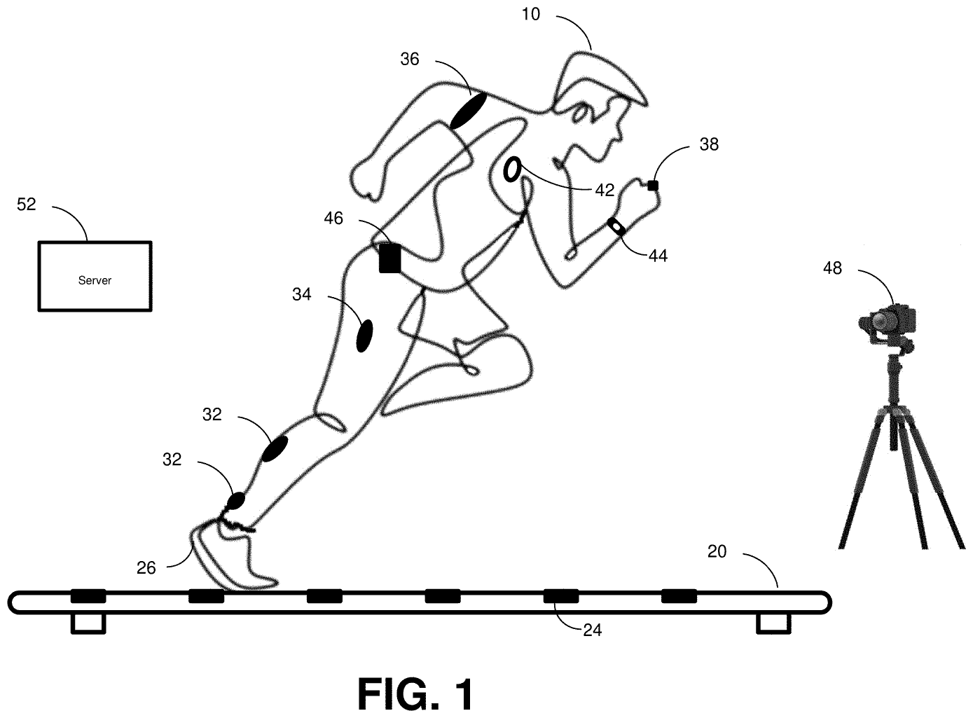

FIG. 1 illustrates an arrangement of various sensing devices (“sensors”) configured to obtain various biomechanical metrics of a human runner 10 (or “subject”), the obtained biomechanical metrics generally including metrics of consideration in the biomechanical and physiological analyses described in this detailed description.

Although this section of this detailed description is intended to provide examples of sensors that can be involved in biomechanical and/or physiological analyses, it should be understood that not all of the sensors depicted in FIG. 1 need be present. Particularly, as should be understood from this detailed description, a first one or more metrics measured by a first one or more sensors may be used to determine or model a second one or more metrics that would otherwise be measured by a second one or more sensors, and accordingly, the second one or more sensors can be omitted. Moreover, in some embodiments, additional and/or alternative sensing devices may be used.

In FIG. 1, the runner 10 is depicted as running along a substantially flat surface 20 (or “ground”), which may be a natural ground, a synthetic track, a moving treadmill, or another surface. Although the substantially flat (i.e., level) surface 20 is depicted for simplicity with regard to consideration of running parameters, it should be understood that a non-level running surface (i.e., having an elevation gradient) could be accounted for, in some embodiments. Moreover, it is presumed for simplicity that the surface 20 is a same surface across of each of two or more running sessions (e.g., races) performed by the runner 10. However, in various embodiments, variations in the type of surface 20 can be during and/or between running sessions can be accounted for.

In some embodiments, e.g., where the running surface 20 is located in a controlled environment, the surface 20 is outfitted with force plates 24 configured to measure vertical, horizontal, mediolateral, and/or combined force exerted through a foot 26 of the runner 10 each time the foot 26 (i.e., foot or shoe) contacts the running surface 20. Generally speaking, a vertical ground reaction force (vGRF) follows approximately an inverse parabolic curve for a duration of time (a “contact time”) during which the foot 26 is in contact with the surface 20 (this duration of time is referred to as “stance”). That is, the vGRF generally increases from zero at first contact to a maximum force as the runner 10 “springs” from the surface 20, and ultimately decreases to zero as the foot 26 launches from the surface 20 and is no longer in contact with the surface 20. The time at which the foot 26 exits contact with the surface 20 begins a “flight time” that lasts until the other foot of the runner 10 contacts the surface 20.

A variety of sensing devices may be placed in contact or proximate to the runner 10. The description of the following sensing devices is not intended to be limiting, as the types, placements, form, and/or functionalities of these sensing devices may vary in accordance with the needs of the runner 10 and/or the measurement(s) sought by an observer. Moreover, still other sensor types may be used, in various embodiments. Sensor nodes 32 are affixed to the upper and lower calf of the runner 10 and may be configured to, for example, measure temperature and/or electrical impulses associated with firing of the muscles of the upper and lower calf. A node 34 may be configured to perform similar functionality with respect to the upper leg of the runner 10. An armband 36 may be configured to, for example, measure blood pressure, heart rate, temperature, and/or blood glucose content of the runner 10. A pulse oximeter 38 may be connected to a finger of the runner 10 and may be configured to measure blood oxygen saturation of the runner 10. Still additionally or alternatively, one or more thoracic sensor nodes 42 may be affixed to the chest of the runner 10, and may be configured for example to detect heart rate, breathing rate, breathing volume, and/or heartbeat rhythms of the runner 10.

In preferred embodiments, a smart wearable device 44 (e.g., smartwatch) is affixed to the runner 44 and is configured to obtain various metrics of the runner 10 during one or more running sessions. For example, the smart wearable device 44 may be configured to measure the runner's wrist-based heart rate, respiration rate, and/or blood oxygen saturation. The smart wearable device 44 may include an inertial measurement unit (IMU, e.g., including an accelerometer and/or gyroscope) configured to measure change in velocity of the runner 10 in the X, Y, and/or Z directions, which may be used to identify steps taken by the runner 10 in connection with an arm/wrist movement cycle associated with each step (e.g., using a native “step counter” application of the smart wearable device 44, or by receiving and analyzing the accelerometer/gyroscope elsewhere). Additionally or alternatively, the runner 10 may wear a hip-mounted sensing device 46 (e.g., a smart wearable device), which may similarly include an IMU configured to measure change in X, Y, and/or Z velocities of the runner 10 to identify steps taken by the runner. Step counting via the hip-mounted IMU may be more reliable than the IMU of the smart wearable device 44, as movement of the hip-mounted sensor 46 will generally correlate strongly to the movement of the center-of-mass of the runner 10. In any case, number of steps can be evaluated over time to determine instantaneous or average step frequency. Additionally or alternatively, the number of steps and/or the step frequency may be evaluated over distance to determine instantaneous or average stride length. Distance traveled may be measured, for example, via a location unit (e.g., GPS unit) of one or more of the sensors carried by the runner 10. The location unit may, for example, measure location of the runner 10 as a function of time, which may be used to determine instantaneous speed of the runner 10 between any two location readings or an average speed of the runner 10 over a longer distance traveled. In some embodiments, the smart wearable device 44 and/or hip-mounted sensor 46 may include an altimeter configured to repeatedly collect elevation readings during a running session, which may similarly be utilized to determine elevation between any two or more steps of the runner 10 and/or an elevation change over a greater distance traveled by the runner 10. In some embodiments, data obtained via the location unit and/or altimeter may be utilized to determine a surface or terrain type used by the runner 10 during one or more running sessions.

Preferred embodiments may use the smart wearable device 44 and/or hip-mounted sensor 46 without many (or any) of the other sensing devices depicted in FIG. 1, at least because the smart wearable device 44 may be less invasive and/or impeding to the typical motions of the runner 10. Furthermore, the smart wearable device 44 and/or hip-mounted sensor 46 may be more easily and consistently available to the runner 10 over a plurality of running sessions during which biomechanical/physiological metrics are to be obtained, e.g., during daily or weekly running within the normal routine of the runner 10.

The sensors of FIG. 1 may include one or more imaging devices 48 (hereinafter “cameras”), which may include one or more photographic camera, one or more thermal cameras, and/or another suitable one or more camera(s). The one or more cameras 48 may be positioned above, below, in front of, behind, and/or alongside of the runner 10. Images obtained by the one or more cameras 48 may be used to for example, directly or indirectly determine the runner's 10 step frequency, stride length, velocity, leg length, touchdown angle, contact time, flight time, elevation change, and/or other suitable biomechanical/physiological metrics described herein. In some embodiments, determination of one or more biomechanical and/or physiological metrics is performed via one or more trained machine learning models configured to extract such metrics from images captured by the one or more cameras 48.

Generally speaking, one or more further computing devices (e.g., servers, not depicted) may be configured to obtain sensed data via the sensors, and analyze the sensed data in accordance with any of the techniques described herein. The one or more further computing devices may obtain the sensed data, for example, via wired and/or wireless signal communications with the sensors using any suitable communication protocol(s) as the sensors obtain sensed data. Additionally or alternatively, sensors of FIG. 1 can be configured to record data via fixed or removable computer storage local to the sensors, and may be wiredly or wirelessly connected to the one or more further computing devices subsequent to data collection. Still alternatively, where sensors of FIG. 1 contain removable computer storage, said removable computer storage may be removed from the sensors subsequent to data collection and provided to the one or more computing devices.

As will be understood from subsequent sections of this detailed description, metrics sensed by the sensors of FIG. 1 or inferred from the sensor data can be supplied to various mathematical models for various purposes, including to model and analyze variations in biomechanical and/or physiological metrics of the runner 10 within a single running session, and/or over two, three, four, or more running sessions. These metrics and variations thereof may have various applications, including to identify potential improvements to performance of the runner 10 (e.g., speed, ability, or energy efficiency), identify individualized patterns and unique characteristics of biomechanical/physiological metrics defining the runner 10, and/or mitigate risk of acute and/or stress-induced injuries to or changes in health status of the runner 10. These analyses may be performed, for example, at a server 52, which may be configured for example to receive data from the sensing devices of FIG. 1 via any suitable one or more of the techniques described herein. Generally speaking, actions of the server 52 may be performed via one or more processors of the server 52 executing non-transitory instructions stored at one or more computer readable memory of the server 52, the instructions configured to cause the server 52 to perform actions described herein.

### Fundamentals of the Spring-Mass Inverted Pendulum (Slip) Model

The human running gait has been described as a “complex dynamical system” of hundreds of variables, with the various muscles, tendons, and joints of the body all contributing and interacting with each other to produce the running gait (Alexander, R. M. (1995). Simple Models of Human Movement. Applied Mechanics Reviews, 48(8), 461-470; Hamill, J., van Emmerik, R. E., Heiderscheit, B. C., & Li, L. (1999)). A dynamical systems approach to lower extremity running injuries. Clinical Biomechanics, 14(5), 297-308). This complexity has challenged the biomechanics field, and there is no consensus definition on what constitutes “healthy” or “optimal” gait (Davis, I. S., & Futrell, E. (2016). Gait Retraining: Altering the Fingerprint of Gait. Physical Medicine and Rehabilitation Clinics of North America, 27(1), 339-355; Heiderscheit, B. C. (2011). Gait retraining for runners: in search of the ideal. Journal of Orthopaedic & Sports Physical Therapy, 41(12), 909-910; Moore, I. S. (2016). Is There an Economical Running Technique? A Review of Modifiable Biomechanical Factors Affecting Running Economy. Sports Medicine, 46(6), 793-807).

A strategy to tackle this challenge of the “complex, high-dimensional, nonlinear, dynamically coupled interactions” in gait is to reduce the dimensionality of the system (i.e., the runner) and study the features fundamental to producing the outcome (Full, R. J., & Koditschek, D. E. (1999). Templates and anchors: neuromechanical hypotheses of legged locomotion on land. Journal of Experimental Biology, 202 (Pt 23), 3325-3332). Alexander similarly proposed that using simple models to study gait would make it “easier to discover which of its [the model's] features are essential to the observed effect” (Alexander, R. M. (1995). Simple Models of Human Movement. Applied Mechanics Reviews, 48(8), 461-470). By studying gait from a template perspective, the redundancies of the myriad biomechanical degrees-of-freedom, from the dozens of force vectors, the hundreds of joint angles and moments, and the millions of muscle cell contractions, are collapsed into a coordinated system. One is then able to study those essential features of the task and their interactions, and any deviations from the template are brought into relief for further investigation.

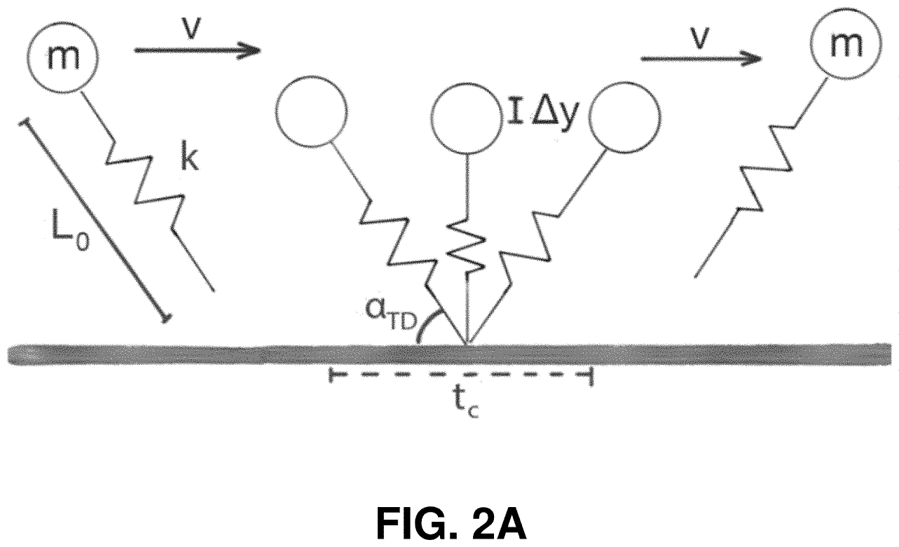

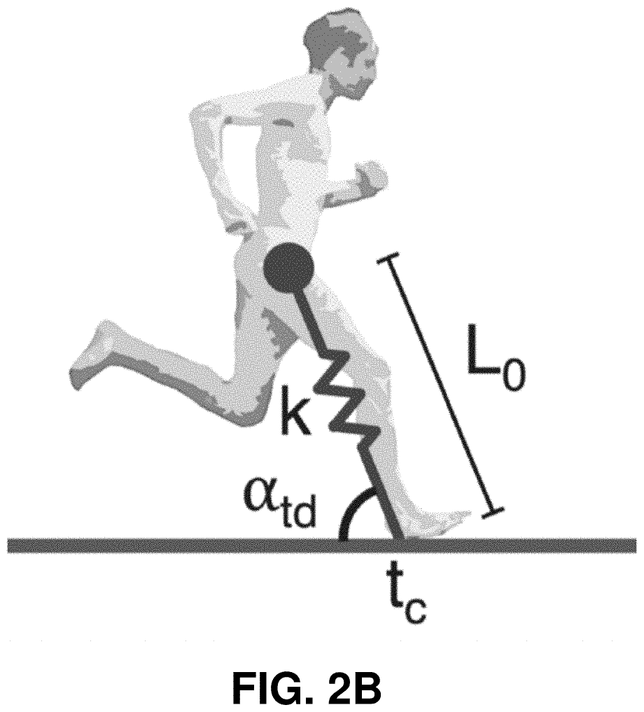

Many templates with varying degrees of complexity have been proposed to model the bouncing gait of running. Multiple historical models were eventually combined into a single model referred to as the spring-loaded inverted pendulum (SLIP) (Blickhan, R. (1989). The spring-mass model for running and hopping. Journal of biomechanics, 22(11-12), 1217-1227; McMahon, T. A., & Cheng, G. C. (1990). The mechanics of running: how does stiffness couple with speed? Journal of biomechanics, 23 Suppl 1, 65-78). This two-dimensional model treats the body as a single point mass on a linearly elastic spring, and it compresses and decompresses in an inverted pendular motion during stance. This SLIP template is also commonly referred to as the spring-mass model of running, and is illustrated in FIG. 2A in the abstract and in FIG. 2B with specific reference to a human runner.

For a given mass (m) and velocity (v), the dynamics of the SLIP system are fully described by only four parameters, as shown in Table 1 below (see Blickhan, 1989; Ludwig, C., Grimmer, S., Seyfarth, A., & Maus, H. M. (2012). Multiple-step model-experiment matching allows precise definition of dynamical leg parameters in human running. Journal of biomechanics, 45(14), 2472-2475).

TABLE 1
SLIP parameters for a given mass and velocity

| Parameter | Description |
|---|---|
| k | Leg Stiffness |
| α_{TD} | Touchdown Angle |
| L₀ | Leg Length |
| t_{c} | Contact Time |

During a flight phase before stance, the system (e.g., a human runner) follows a projectile motion subject only to gravity (g), with the motion of the system's x-y center-of-mass (COM) being described by Equations 1 and 2 in the list of equations provided below. During stance, the system touches down and remains fixed at xo, and the system COM motion is described by Equations 3, 4, and 5 below, with Equation 5 denoting the length L of the leg spring throughout stance.

$$\frac{{d}^{2} x}{{dt}^{2}} = 0 \qquad 1$$

$$\frac{{d}^{2} y}{{dt}^{2}} = - g \qquad 2$$

$$\frac{{d}^{2} x}{{dt}^{2}} = \frac{k}{m} ( {L}_{0} - L ) ( \frac{x - {x}_{0}}{L} ) \qquad 3$$

$$\frac{{d}^{2} y}{{dt}^{2}} = \frac{k}{m} ( {L}_{0} - L ) ( \frac{y}{L} ) - g \qquad 4$$

$$L = \sqrt{{y}^{2} - {( x - {x}_{0} )}^{2}} \qquad 5$$

$$Equations 1 , 2 , 3 , 4 , and 5 - SLIP dynamics$$

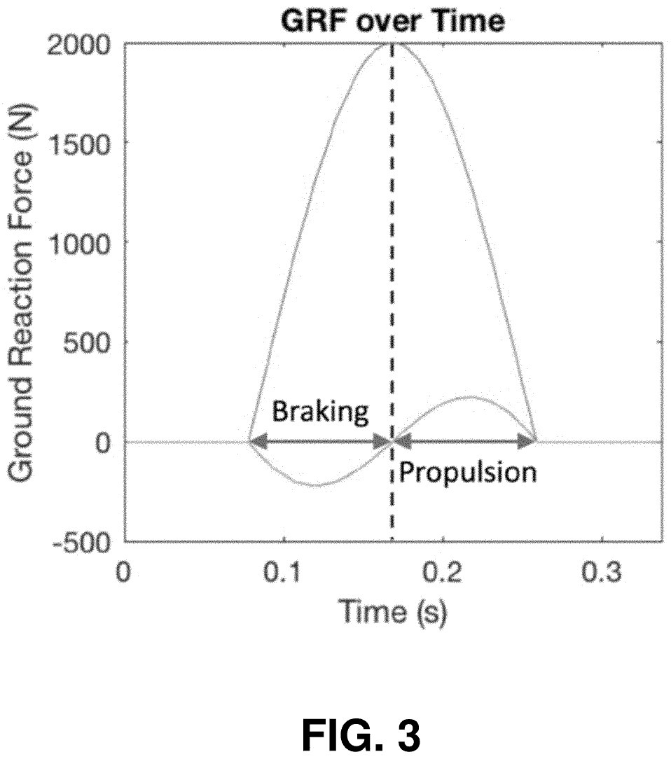

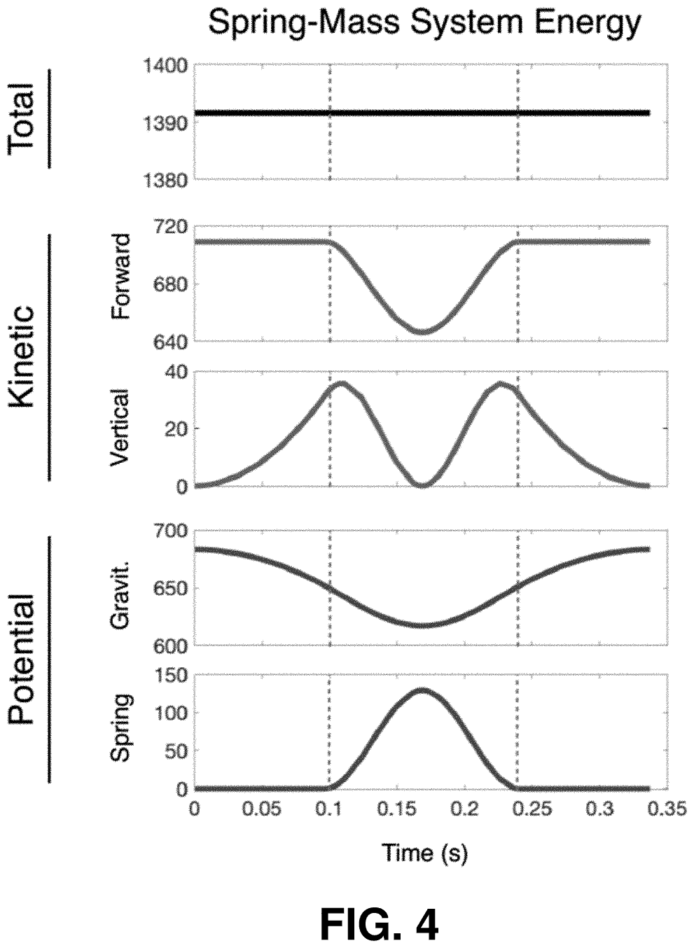

The behavior described above results in a coordinated transfer of energy throughout the gait cycle. The horizontal GRF has an equal and opposite braking and propulsive impulse, with the cross-over of the force occurring at midstance. The vertical GRF is symmetric, with the peak also occurring at midstance, as illustrated in FIG. 3. The vertical COM displacement inversely follows the vertical GRF and reaches its minimum at midstance. As such, the system is energy-conservative, and the changes in vertical and horizontal kinetic energy and elastic and gravitational potential energy through a step cycle occur in phase, as illustrated in FIG. 4 with respect to an example model having a mass of 70 kilograms (kg) with a 1.0 meters (m) leg moving at 4.5 meters second (m/s).

The SLIP model is most widely used as a descriptive tool to calculate the “stiffness” of the runner. There are several approaches to this calculation, and they are derived from the theoretical relation of the model's spring stiffness to its change in spring length under maximal vertical force at midstance. The two most common methods used in gait research to calculate stiffness come from McMahon and Cheng (see McMahon & Cheng, 1990) and Morin et al. (Morin, J. B., Dalleau, G., Kyrolainen, H., Jeannin, T., & Belli, A. (2005). A simple method for measuring stiffness during running. Journal of Applied Biomechanics, 21(2), 167-180). Both of these methods differentiate vertical stiffness (kᵥₑᵣₜ) and leg stiffness (k_{leg}) of the runner to characterize behavior of vertical displacement and leg length changes during the gait cycle under maximal force. As a dynamic SLIP model functions with a single linear elastic spring, the description here will be restricted to that of leg stiffness calculations. McMahon and Cheng described k_{leg} of a SLIP model as the ratio of maximal vertical force, Fₘₐₓ, and maximal spring-leg displacement, ΔL.

$${k}_{leg} = \frac{{F}_{ma x}}{Δ L} \qquad 6$$

$$Δ L = Δ y + {L}_{0} ( 1 - sin {α}_{TD} ) \qquad 7$$

$${α}_{TD} = {cos}^{- 1} \frac{v {t}_{c}}{2 {L}_{0}} \qquad 8$$

$$Equations 6 , 7 , and 8$$

This method relies on recording a step with a force plate to measure Fₘₐₓ and t_{c} (McMahon & Cheng, 1990). The vertical force recording is also used to calculate Δy, the COM displacement, via double-integration of the vertical force (Cavagna, G. A., Heglund, N. C., & Taylor, C. R. (1977). Mechanical work in terrestrial locomotion: two basic mechanisms for minimizing energy expenditure. American Journal of Physiology, 233(5), R243-261). The method also relies on measurement of L₀ from the runner, often taken as the distance from the greater trochanter to the floor while standing (Brughelli, M., & Cronin, J. (2008). A review of research on the mechanical stiffness in running and jumping: methodology and implications. Scandinavian Journal of Medicine & Science in Sports, 18(4), 417-426; McMahon & Cheng, 1990) or as a ratio of 0.53 to the standing height of the runner (Morin et al., 2005). The method estimates □_{TD} m per Equation 8 above. McMahon and Cheng compared dynamic measures, such as stride length, duty factor, and contact time, across running speeds from a SLIP-simulation and from experimental observations of a runner. They concluded there was generally good agreement, though no system validation was conducted (McMahon & Cheng, 1990).

The method proposed by Morin and his colleagues does not rely on a force plate for measurement but rather approximates the vertical GRF as a sinusoid using the ratio of t_{c} and the flight time, t_{f}, to estimate Fₘₐₓ and Δy. This method requires measurement of the contact time, flight time, running speed, and resting leg length. They compared the vertical impulse, the area under the vertical GRF time curve, of their sinusoid-approximated SLIP model to that of the observed vertical impulse and found a bias of 5.33% and 2.93% in treadmill and overground running, respectively (Morin et al., 2005). Other methods have been used to estimate leg stiffness (e.g., Arampatzis, A., Bruggemann, G. P., & Metzler, V. (1999). The effect of speed on leg stiffness and joint kinetics in human running. Journal of biomechanics, 32(12), 1349-1353; Cavagna, G. A., Franzetti, P., Heglund, N. C., & Willems, P. (1988). The determinants of the step frequency in running, trotting and hopping in man and other vertebrates. Journal of Physiology, 399, 81-92; McMahon, T. A., Valiant, G., & Frederick, E. C. (1987). Groucho running. Journal of Applied Physiology, 62(6), 2326-2337), but they are less commonly applied.

The SLIP template has also been applied as a predictive tool for certain biomechanical parameters of gait. Stance time, aerial time, duty factor, vertical COM displacement, horizontal COM excursion during stance, vertical impulse, and horizontal impulse are all common measures that can be estimated with observed GRF recordings informing SLIP model simulations (Bullimore, S. R., & Burn, J. F. (2007). Ability of the planar spring-mass model to predict mechanical parameters in running humans. Journal of Theoretical Biology, 248(4), 686695). Bullimore and Burn simulated SLIP running with experimentally observed GRF recordings by calculating k_{leg} from Equations 6-8 and assigning it to the model constrained to behave like a stable SLIP system (e.g., symmetric energy fluctuations). They compared ten common gait measures predicted by the simulated SLIP system with the observed values in the runners. There was generally good agreement between modeled and observed gait, with modest overestimation (%) in duty factor (6.8%), stride length (3.6%), peak vertical GRF (3.8%), t_{c} (2.5%), horizontal excursion during stance (5.1%), and vertical impulse (5.8%). However, larger errors were observed in flight time (17.7%), vertical COM displacement (22.9%), horizontal impulse (43.6%), and peak mechanical energy change during stance (26.2%) (Bullimore & Burn, 2007). Rather than using single parameter comparisons, Seyfarth and colleagues evaluated the quality of SLIP model parameter combinations by assessing the number of “steps” the simulated model would take before becoming unstable (Seyfarth et al., 2002). Using this method, Blum and coworkers evaluated several methods of estimating k_{leg} across speeds of 2-4 m/s, using measured values of L₀, □_{TD}, and v (Blum, Y., Lipfert, S. W., & Seyfarth, A. (2009). Effective leg stiffness in running. Journal of biomechanics, 42(14), 2400-2405). The k_{leg}-□_{TD} combinations were mapped across those of a stable SLIP system (determined as one that could take more than four consecutive steps without becoming unstable) to qualitatively compare the experimental observations. The sinusoid approximation of SLIP parameters and a duty-factor approximation of the parameters were concluded to yield generally stable gait cycles, but the effect of speed was not evaluated (Blum et al., 2009).

### Challenges and Developments in Slip Modeling of Runners

Direct model-experiment comparison of full COM trajectories and GRF time curves has been limited. Performing this analysis from purely kinetic recordings is challenging given the aforementioned computational complexity of the model. One means that has been employed is that of assessing the force-length curve of the modeled leg spring. Gunther and Blickhan demonstrated a means to do this by fitting slopes to the force-length curve during both compression and decompression (Gunther, M., & Blickhan, R. (2002). Joint stiffness of the ankle and the knee in running. Journal of biomechanics, 35(11), 1459-1474). The challenge of this method lies in the hysteresis in the compression and decompression, where significant deviations from linearity in early and late stance bias the fit. They attempted to resolve this by fitting a linear function with a state-shift for the compression and decompression periods, through this still underestimated leg compression. They also attempted to address the shift by fitting the force-length relationship with a nonlinear term on the leg-length change, but this was similarly subjected to significant deviations driven by the distal accelerations. Importantly, this method also relied on kinematic inputs from motion capture equipment, which itself has anthropometric assumptions of the center-of-mass for marker location (taken as the hip here) and leg length compression. It is also subject to initial condition assumptions for the computation and force-length fitting. The estimates of leg stiffness from the method were thus sensitive to initial conditions of the leg coordinates as well as those of each of the velocity components (Gunther & Blickhan, 2002).

Lipfert and colleagues compared leg-force-length curves calculated directly from the GRF time curve to those of a SLIP model with estimated k_{leg} values and measured L₀ and □_{TD} values (Lipfert, S. W., Gunther, M., Renjewski, D., Grimmer, S., & Seyfarth, A. (2012). A model-experiment comparison of system dynamics for human walking and running. Journal of Theoretical Biology, 292, 11-17). They calculated the coefficient of determination between the model-based and experimental curves and found good agreement that was independent of running speed (R²=0.94-0.99). However, this value was modified by the difference between simulated and experimentally observed t_{c} values, and the simulations were unable to produce stable gait cycles with the estimated k_{leg} and measured □_{TD} values, so □_{TD} was adjusted in the simulations to generate stable solutions (Lipfert et al., 2012). Ludwig et al. compared SLIP simulations to a single runner across several steps to evaluate the ability of a passive or actuated SLIP model to match human running gait (Ludwig et al., 2012). They estimated k_{leg}, □_{TD}, and L₀ as those which characterized a stable SLIP system that matched the observed maximal COM height, minimal COM height, and t_{c}. The SLIP systems and experimental observations were compared across many gait parameters, with Fₘₐₓ being slightly underestimated in the SLIP system (2.36 BW vs. 2.53 BVV) and the step length being slightly overestimated (1.043 m vs. 1.037 m) for the first step (Ludwig et al., 2012).

Similar to the challenges described above in the estimation of parameters from model experiment tracking, simply comparing the degree to which a runner behaves like a SLIP system is difficult. One way to quantify this is via the hysteresis of the compression and decompression of the leg described above. This requires kinematic recording of the assumed leg (e.g., from the hip), and it is therefore sensitive to the spatial and dynamic initial conditions assumed (Gunther & Blickhan, 2002). For simplicity, investigators sometimes choose to present force-displacement curves from the vertical component alone—i.e., the vertical compression of the leg spring. This is problematic for two reasons. The first is that the compression of the SLIP spring does not happen purely in the vertical plane—its assumption of linear elasticity occurs in dynamic, pendular motion (Blickhan, 1989). As such, modeling vertical compression as a Hookean spring is incorrect and will consequently demonstrate nonlinearity. Second, this approach is heavily biased by deviations in the early and late phases of stance where the magnitudes of center-of-mass vertical displacement are greater for relatively low magnitudes of force. A common deviation in this period is the impact peak, where its contribution results in distinct compression and decompression force-displacement curves. Dutto et al. observed several centimeters of variation between initial and final positional estimates from this (Dutto, D. J., & Smith, G. A. (2002). Changes in spring-mass characteristics during treadmill running to exhaustion. Medicine & Science in Sports & Exercise, 34(8), 1324-1331), and Hunter demonstrated that fitting a separate stiffness term for the impact peak would partially reconcile the poor fit (Hunter, I. (2003). A new approach to modeling vertical stiffness in heel-toe distance runners. Journal of Sports Science and Medicine, 2(4), 139-143). Cavagna and Legramandi attempted to reconcile this by calculating the hysteresis during the period that the runner exceeded body weight, termed the “effective contact time” of stance (Cavagna, G. A., & Legramandi, M. A. (2015). Running, hopping and trotting: tuning step frequency to the resonant frequency of the bouncing system favors larger animals. Journal of Experimental Biology, 218 (Pt 20), 3276-3283). While this resolves the distinct nonlinearity often observed in early and late stance, and thus is less subject to bias from deviations in those periods (though not free from it), it still assumes vertical linear elasticity. When this hysteresis is assessed as a ratio or percentage of the integrated compression and decompression force-displacement periods, it does hold true that the spring-mass system will maintain a unity value of 1.0 given its conservative nature. However, because the vertical compression is not strictly linearly elastic, deviations at different points along the curve will bias the estimate non-uniformly.

Another method to calculate the degree to which a runner behaves like a spring-mass system is derived from the takeoff-landing asymmetry observations of Cavagna (Cavagna, G. A. (2006). The landing-take-off asymmetry in human running. Journal of Experimental Biology, 209 (Pt 20), 4051-4060). He examined the energetic fluctuations of runners' center-of-masses through the gait cycle via force plate recordings and calculated the timing characteristics. Two quantities he noted were the braking and pushing durations, which correspond to the periods of decelerating and accelerating the center-of-mass, and the ratio of the maximal downward velocity and maximal upward velocity. In a perfectly elastic, energy-conserving system, such as the SLIP system, these two quantities should be equal. He and Legramandi formalized this as a metric they termed the “similarity to a symmetric bounce”, where they calculated the average of the two quantities (Cavagna & Legramandi, 2015). They previously demonstrated that these two quantities peaked in teenage runners and slowly declined with age (Legramandi, M. A., Schepens, B., & Cavagna, G. A. (2013). Running humans attain optimal elastic bounce in their teens. Scientific Reports, 3, 1310).

Though the SLIP template has been used for decades to describe the elastic nature of gait, there are many limitations and shortcomings in the conventional approaches to its application. The first of these is in the measurement of model parameters. With a known velocity, the dynamics of the SLIP model are determined by only four parameters (Table 1), yet in all forms of gait analysis, some of these parameters are necessarily assumed or assigned values from empirical observation. That is, the SLIP model is constrained to that specific parameter. The most common of these is the leg length, L₀, of the system. In the SLIP model, this is the distance from the center-of-mass to the point of contact on the ground. However, the parameter is nearly always assigned the value of the standing leg length (e.g., greater trochanter to the ground) (Brughelli, M., & Cronin, J. (2008). A review of research on the mechanical stiffness in running and jumping: methodology and implications. Scandinavian Journal of Medicine & Science in Sports, 18(4), 417-426; Bullimore & Burn, 2007; McMahon & Cheng, 1990) or as a COM anthropometric assumption based on height, h, where L₀=0.53 h (Morin et al., 2005). In reality, a human's center-of-mass is difficult to determine statically (Clauser, C. E.; McConville, J. T.; Young, J. W. Weight, Volume, and Center of Mass Segments of the Human Body, Journal of Occupational Medicine: May 1971-Volume 13-Issue 5-p 270), and quite complex dynamically, and almost certainly not defined by the length of the human leg (Kingma, I., Toussaint, H. M., Commissaris, D. A., Hoozemans, M. J., & Ober, M. J. (1995). Optimizing the determination of the body center of mass. Journal of biomechanics, 28(9), 1137-1142; Maus, H. M., Seyfarth, A., & Grimmer, S. (2011). Combining forces and kinematics for calculating consistent center of mass trajectories. Journal of Experimental Biology, 214(21), 3511-3517; Naga, S. (2005). An Efficient Algorithm for Clinical Mass Center Location of Human Body. Nakayama, Y., Kudo, K., & Ohtsuki, T. (2010). Variability and fluctuation in running gait cycle of trained runners and non-runners. Gait & Posture, 31(3), 331-335; Saini, M., Kerrigan, D. C., Thirunarayan, M. A., & Duff-Raffaele, M. (1998). The vertical displacement of the center of mass during walking: a comparison of four measurement methods. Journal of Biomechanical Engineering, 120(1), 133-139). Clauser and colleagues measured the COM ratio to height as being 0.58, which would yield a 7-10 cm difference in L₀ estimation (Clauser et al., 1969) from the traditional 0.53 assumption. This 10% difference in L₀, based solely on which COM assumption is adopted, would yield a 7% difference in k_{leg} estimation (Morin et al., 2005). The SLIP model is highly sensitive to changes in its parameters, so small fluctuations in parameter inputs can negatively affect its stability. Indeed, different assumptions of the leg length will inevitably change estimations of the model parameters (Brughelli & Cronin, 2008; Morin et al., 2005) and affect the ability to generate stable parameter combinations (Lipfert et al., 2012). Only one investigation using a 2D SLIP template estimated L₀ from observed gait data, and it was a case study with a single subject (Ludwig et al., 2012).

Similarly, □_{TD} is frequently calculated from Equation 8 (Brughelli & Cronin, 2008; Bullimore & Burn, 2007; McMahon & Cheng, 1990; Morin et al., 2005) or measured kinematically as the angle of the leg at touchdown (Blum et al., 2009). Equation 8 necessarily underestimates the angle, as the velocity during stance must be lower than the gait cycle's average velocity (per Equations 1-4). Kinematic measurement resolves this, but itself carries the center-of-mass positional assumption (and those associated with the motion capture processing). Even more so than with L₀ fluctuations, the SLIP template parameters vary greatly across a small range of □_{TD} (Seyfarth, A., Geyer, H., Gunther, M., & Blickhan, R. (2002). A movement criterion for running. Journal of biomechanics, 35(5), 649-655). Fractions of a degree can yield great discrepancies in stiffness estimates and fail to enable a model to achieve stable gait (Lipfert et al., 2012).

Finally, t_{c} is nearly always constrained to that of the observed t_{c} (Brughelli & Cronin, 2008). Though this seems logical given that it is directly observable, unlike L₀ or □_{TD}, it can be problematic if one is trying to describe a SLIP system that best fits a runner. There is a tendency towards asymmetry in the stance phase of gait, with the end of the cycle often displaying a deviation from linear elasticity in the GRF curve (Cavagna, 2006; Cavagna, G. A., Legramandi, M. A., & Peyre-Tartaruga, L. A. (2008). The landing-take-off asymmetry of human running is enhanced in old age. Journal of Experimental Biology, 211 (Pt 10), 1571-1578; Clark, K. P., Ryan, L. J., & Weyand, P. G. (2017). A general relationship links gait mechanics and running ground reaction forces. Journal of Experimental Biology, 220 (Pt 2), 247-258). Even if this occurs in the final one or two hundredths of a second during stance, constraining the model to match this complete time course may bias description of the true elastic behavior of the runner and provide an inaccurate representation of the SLIP template. Indeed, allowing t_{c} to vary between model and experiment elicits more accurate predictions of COM trajectory (Lipfert et al., 2012). Burns et al. demonstrated a means to estimate all four parameters simultaneously from observed data using a novel nonlinear regression method, and the parameters that were estimated from the novel method generated more stable SLIP simulations and better modeled the observed vGRF curves of runners (Burns, G. T., Gonzalez, R., & Zernicke, R. F. (2021). Improving spring-mass parameter estimation in running using nonlinear regression methods. The Journal of experimental biology, 224 (Pt 6), jeb232850. https://doi.org/10.1242/jeb.232850).

As such, each of these metrics as calculated are co-dependent to some degree, as leg stiffness is traditionally calculated using the leg length change approximation in Equation 7, which relies not only on the leg length assumption, but on kinematic assumptions for spring-leg position or the touchdown angle approximation in Equation 8. That itself is not only a constant-velocity approximation, but it also relies on and is constrained to the observed contact time. Errors in those assumptions can therefore be manifested in and affect all parameters. Misrepresenting contact time, for example, will propagate the error throughout the touchdown angle and the final stiffness value.

One of the significant challenges in analyzing a runner as a SLIP system is the computational complexity of the model (Robilliard, J. J., & Wilson, A. M. (2005). Prediction of kinetics and kinematics of running animals using an analytical approximation to the planar spring-mass system. Journal of Experimental Biology, 208 (Pt 23), 4377-4389). Optimization techniques are routinely used in biomechanics investigations to estimate model parameters from experimentally observed data (Robertson, G. E., Caldwell, G. E., Hamill, J., Kamen, G., & Whittlesey, S. (2018). Research Methods in Biomechanics: Human kinetics). However, the optimization and simulation methods required to generate stable models while tracking experimental data are computationally intensive, and model-experiment analyses have therefore been restricted to a limited number of steps or subjects (Blickhan, 1989; Bullimore & Burn, 2007; Lipfert et al., 2012; Ludwig et al., 2012; Seyfarth et al., 2002; Seyfarth, A., Geyer, H., & Herr, H. (2003). Swing-leg retraction: a simple control model for stable running. Journal of Experimental Biology, 206 (Pt 15), 2547-2555). Moreover, numerical iteration to solve best-fit parameter combinations do not necessarily reveal mechanisms for interaction among the gait parameters. To resolve the computational complexity, the SLIP model GRF has been approximated by a sinusoidal function. These models have demonstrated good agreement in stiffness estimation and GRF impulse characterization with the traditional model (Blum et al., 2009; Morin et al., 2005; Robilliard & Wilson, 2005). However, they have not been used for simultaneous multi-parameter estimation or model-experiment comparison across the entire GRF curve or COM trajectory.

Single-subject investigations of SLIP template comparisons have revealed a high degree of within-subject, step-to-step variability in parameters (Blum et al., 2009; Lipfert et al., 2012; Ludwig et al., 2012; McMahon & Cheng, 1990; Seyfarth et al., 2002). However, it is common across gait biomechanics studies to collapse many gait cycles to ensemble averages or analyze factors in isolation (Ferber, R., Osis, S. T., Hicks, J. L., & Delp, S. L. (2016). Gait biomechanics in the era of data science. Journal of biomechanics, 49(16), 3759-3761). Indeed, use of the SLIP template is often reduced to calculating single leg stiffness values for subjects without consideration to covariance with the other parameters or time-variance within the analysis.

In view of at least these challenges and the limitations in the scope of the existing research, Burns, Gonzalez, and Zernicke established analytical and computational frameworks to study runners against a SLIP template, and presented additional applications of the framework to deliver template-derived biomechanical insights (see Burns, G. T., Gonzalez, R., & Zernicke, R. F. (2021). Improving spring-mass parameter estimation in running using nonlinear regression methods. The Journal of experimental biology, 224 (Pt 6), jeb232850. https://doi.org/10.1242/jeb.232850). The work presented a method to model runners as spring-mass systems using nonlinear regression (NLR) and the full vertical ground reaction force (vGRF) time series without additional inputs and fewer traditional parameter assumptions. A time-dependent vGRF function was derived and validated, the function characterized by four spring-mass parameters (stiffness, touchdown angle, leg length and contact time) using a sinusoidal approximation. The work of Burns, Gonzalez, and Zernicke compared the NLR-estimated spring-mass parameters with traditional calculations in runners. The mixed-effect NLR method (ME NLR) modeled the observed vGRF best (RMSE: 155 N) compared with a conventional sinusoid approximation (RMSE: 230 N). Against the conventional methods, its estimations provided similar stiffness approximations (−0.2±0.6 kN m⁻¹) with moderately steeper angles (1.2±0.7 deg), longer legs (+4.2±2.3 cm) and shorter effective contact times (−12±4 ms). Together, these vGRF-driven system parameters more closely approximated the observed vertical impulses (observed: 214.8 N s; ME NLR: 209.0 N s; traditional: 223.6 N s). The work generated spring-mass simulations from traditional and ME NLR parameter estimates to assess the predicative capacity of each method to model stable running systems. In 6/7 subjects, ME NLR parameters generated models that ran with equal or greater stability than traditional estimates. ME NLR modeling of the vGRF in running thus proved to be a useful tool to assess runners holistically as spring-mass systems with fewer measurement sources or anthropometric assumptions (Id. at Abstract). Moreover, by deriving these measures of a runner's spring-mass parameters in this manner, the techniques of Burns, Gonzalez and Zernicke enabled spring-mass parameters to be derived without the conventional reliance upon particular sensor arrangements (e.g., without necessarily relying on sensors that a runner may consider obtrusive or which might be otherwise unavailable to directly measure said parameters). The techniques of Burns, Gonzalez and Zernicke particularly evaluated elite middle-distance runners, evaluating behavior/variance of SLIP parameters and/or other metrics within the observed running session.

Generally speaking, methods described in subsequent portions of this detailed description provide for personalized analysis of the relations between mechanical and physiological variables (VOIs— variables of interest) and their predictors (e.g., speed) and the changes in those relations, both within exercise sessions and across sessions. Additionally or alternatively, VOIs may be analyzed individually, e.g., to identify trends that may correspond to improved performance or injury, or to identify patterns that may be used to uniquely identify a particular runner. These variables of interest can include various ones of the biomechanical metrics described above and/or values obtained indirectly from such metrics, e.g., stride length, stride frequency, heart rate, and/or one or more of the spring-mass parameters in the SLIP model as described above. Although certain techniques for determining/validating SLIP parameters and the advantages thereof have been described in the foregoing, it should be understood that the analysis of biomechanical/physiological metrics (and the variations thereof) described herein is not necessarily limited to metrics identified via any one particular method or framework. For example, where relationships involving a given SLIP parameter (e.g., contact time) regarding a runner are described, the methods described herein may operate upon the parameters as obtained via any of (1) direct or indirect measurement of the runner, (2) a SLIP model of the runner as developed via the conventional techniques described above, (3) a SLIP model of the runner as developed by the NLR and vGRF framework developed by Burns, Gonzalez, and Zernicke, and/or (4) still other techniques/frameworks developed in the past or future.

### Longitudinal Profilng of Athletes Across One or More Running Sessions

Systems and methods described herein provide for personalized analysis of biomechanical and/or physiological behaviors of a single runner across one, two, three, four or more running sessions. Particularly, the personalized analysis examines the relationships between variables of interest (VOIs) and their predictors, both within a single running session and across multiple running sessions. By modeling the relationships of VOIs to predictors, especially continuous predictors, across time both within and across sessions with data selectively managed from ecological collections (e.g., outside the lab in daily living), insights into performance, injury, training adaptation, and external effect attribution can be gained above and beyond traditional approaches or discrete analyses.

Furthermore in some embodiments, VOIs to be analyzed individually, e.g., to identify trends or noise in a VOI during one running session, to examine how trends or degree of noise in the same VOI change over two or more parts of one running session, or examine how the trends or degree of noise in the same VOI differ across two, three, or more running sessions. In some applications, this analysis may be used to identify courses of action to improve performance in a runner, e.g., by determining that a runner's increasing or decreasing variability of a VOI would improve energy economy during part or a whole of a running session. In other applications, these analyses may be used to prevent or mitigate acute or stress-related injury, e.g., by determining that increasing or decreasing a VOI (or increasing/decreasing the stability thereof) would reduce the risk of injury (e.g., by correlation to empirical evidence of runner injury, or by inferring risk of injury e.g., based upon stance forces experienced by the runner). Still additionally or alternatively, in some applications, analysis of one or more VOIs, or relationships between one or more VOIs and one or more predictors, may be used to create a “biomechanical passport” of a given runner by defining the typical values or relationships specific to the runner, and to identify persistence of or changes within the metrics or relationships of the runner from new data.

In various embodiments, VOIs examined via the systems and methods described herein may include, for example, stride length, stride frequency, one or more spring-mass parameters (e.g., stiffness, contact time, and/or touchdown angle), COM displacement during stance, ground reaction force (GRF), heart rate, blood pressure, breathing rate, breathing volume, blood glucose, body and/or skin temperature, and/or blood oxygen saturation. Although an example used herein will consider the relationships of one or more VOIs particularly with respect to speed of the runner, various embodiments of the techniques described in may consider relationships of one or more VOIs to other predictors, which may include other ones of the VOIs discussed herein. Generally, longitudinal analysis of relationships between one or more VOIs and their predictor(s) may comprise a plurality of stages including (1) aggregation and initial processing of raw data from direct measurement sources, (2) transformation and calculation of VOIs from direct measurement sources, (3) elevation gradient appending, (4) data cleaning, (5) single session modeling of the VOI(s) with respect to the predictor(s), and/or (6) multiple session aggregation of the single-session modeling. Each of these steps will be discussed in further detail in the follow sections of this detailed description, and it should be understood that additional, fewer, and/or alternate actions may be involved, in various embodiments.

### Data Aggregation and Initial Processing

A first stage of longitudinal analysis generally involves aggregation of raw data (“running data”) indicative of biomechanical and/or physiological metrics corresponding to a running session. The raw data is aggregated via one or more data sources, which may for example include any one, two, three, or more of the sensing devices described with respect to FIG. 1. In some embodiments, for example, the raw data is collected by the smart wearable device 44 and/or the hip-mounted device 46, either or both of which may be equipped with a positioning unit (e.g., GPS), an IMU, a heart rate monitor, and/or other units configured to measure biomechanical/physiological metrics described herein. In any case, aggregated raw data may effectively include a time series for any particular metric, including values of the metric at a plurality of times during the running session.

Data may be aggregated, for example, by being provided from the one or more data sources to one or more centrally disposed computing devices (e.g., server 52 of FIG. 1) configured to perform the analysis described in this detailed description. Data may be provided from the one or more data sources to the centrally disposed computing device by any technique(s) described herein (e.g., wired and/or wireless communication of data from data sources to the central computing device during or after a running session, or transference of removable memory from data sources and provision of the removable memory to the central computing device).

Initial processing may be performed on the data collected by from the one or more data sources (e.g., processing may be performed at the central computing device(s)). Initial processing may, for example, include data smoothing, filtering, and/or other signal processing of a time series of a collected metric, e.g., by removing obvious outliers or erroneous data points from the time series. In some embodiments, data smoothing includes calculating a moving average of the collected metric (e.g., to form a moving-average curve of the runner's heart rate or another metric over the running session or a portion thereof).

Moreover, in some embodiments, initial processing may involve identifying and extracting a window of data points from the time series for a particular portion of the running session, such that the time series for a metric in a first running session can be appropriately compared to corresponding measures of the same metric in second, third, and/or fourth sessions. For example, a longitudinal analysis of heart rate or stride length over two or more running sessions might only seek to analyze the first 10 km or last 10 km of each running session, regardless of the total distances of the respective running sessions. In some embodiments, multiple sampling windows are evaluated (e.g., the first and last 5 km of each running session are analyzed together or in parallel). An example of this form of data extraction can be found in Burns, G. T., Zendler, J. M., & Zernicke, R. F. (2019). Step frequency patterns of elite ultramarathon runners during a 100-km road race. Journal of applied physiology (Bethesda, Md.: 1985), 126(2), 462-468. https://doi.org/10.1152/japplphysio1.00374.2018, which is hereby incorporated by reference in its entirety.

Still additional and/or alternative forms of initial processing and/or data aggregation may be performed, in accordance with the nature of the collected data set(s) and/or the preferred statistical techniques of the person(s) or machine(s) performing the analyses described herein. In some embodiments, the first stage of data aggregation includes collecting/aggregating subject metadata not directly observed via the sensor(s). For example, it may be obtained from the subject an indication of one or more running surfaces used during the one or more running sessions (e.g., grass, dirt, asphalt, etc.), an indication of one or more shoe models used by the runner during the one or more sessions, a height of the runner, a weight of the runner, and/or a change in weight of the runner during and/or across one or more running sessions. In some embodiments, any of these metadata parameters may be used as predictors and/or VOIs in the analysis described herein, or the metadata parameters may be applied to still other biomechanical calculations or transformations.

### Calculation of Variables of Interest Based Upon Raw Data

In some embodiments, a second stage of longitudinal analysis includes transformation and calculation of the one or more variables of interest (VOIs) from the raw data described in the previous section. As discussed previously herein, one or more first biomechanical/physiological metrics over a time period may be used to obtain a second one or more biomechanical/physiological metrics over the same time period, using simple mathematics and/or more advanced statistical methods. For example, if stride frequency is measured based upon accelerometer data over a sampling window, stride length can be obtained based upon a smoothed distance estimate over the same sampling window. As another example, in embodiments where a particularly high-resolution IMU is included in the sensing device(s), the accelerometer and/or gyroscope data may be used to estimate contact times and/or flight times during the running session (i.e., the amount of time the runner's foot is in contact with the ground or in the air with each step, respectively). Still additionally or alternatively, the same accelerometer data may be used to estimate a ground reaction force (GRF) curve and/or the center-of-mass (COM) displacement of the runner at each step as a time series over the running session. The GRF and/or COM displacement may be used to inform more advanced spring-mass parameter estimations (e.g., leg stiffness) and/or other kinetic measures to be modeled as variables of interest in the longitudinal analyses described herein. Furthermore, in various embodiments, location points (e.g., GPS location) may be attached to each point in a time series, e.g., based upon data from a location unit of a sensing device.

Effectively, based on the understood relationships between two or more metrics, a time series for a given metric over a running session may be obtained even without the presence of a sensing device configured to directly measure the given metric during the running session. If necessary, similar data processing may be performed upon a VOI obtained by these techniques, e.g., to remove erroneous data points in a time series that may be induced by unreliable data points in the metric(s) from which the VOI was obtained.

### Gradient Appending

In some embodiments, a third stage of a longitudinal analysis includes consideration of elevation gradient in the running window from which data was extracted as described above. That is, variables of interest may be subject to differ based upon whether the degree to which the runner was running uphill and/or downhill during one or more portions of the running session, and thus, it would be advantageous to consider elevation gradient at each individual data point in a time series. Elevation gradient for each point a time series may be determined, for example, by overlaying a smoothed geographic elevation raster for the running location to location points (e.g., GPS location points) corresponding respectively to the data points in the time series. Thus, for example, if a heart rate time series is considered, the heart rate at each data point in the time series can be associated with a specific location and an elevation gradient of the specific location. In some embodiments, data points may be automatically adjusted accordingly when a reliable function exists modeling the variable of interest based on elevation gradient.

### Data Cleaning

In some embodiments, a fourth stage of longitudinal analysis includes filtering the data produced at the preceding stage(s) based upon relevant and physiologically realistic ranges for one or more variables of interest and/or one or more predictors. For example, in some scenarios, erroneous data points associated with a measurement of a variable of interest, may produce values known to not be feasible in a human runner (e.g., a heart rate above 300 beats-per-minute or below 15 beats-per-minute, or a stride length above 3 meters). Alternatively, in some scenarios, a produced value may be physiologically realistic but not within a range of values being analyzed (e.g., anomalous data points may be removed if those data points are associated with a brief, one-off period of overexertion or underexertion during the running session).

### Single-Session Modeling

A fifth stage of longitudinal analysis may generally include, for each running session individually, building a model of the relationship of one or more VOIs to one or more predictors based upon the data collected from the respective running session. In particular, this stage may include determining the beta (β) coefficients defining a general linear model (and more particularly, a multiple regression model) of the relationship between the VOI(s) and a predictor. Upon generating the model, residual plots (e.g., corresponding to subsequent running sessions) may be used to verify the generated model and/or examine the generated model for presence of underlying biasing phenomenon. If residual plots and/or further examination indicate error or deficiencies in the model, the model may be iteratively adjusted to account for the error or deficiencies.

In an example to be provided in this section with respect to equations and accompanying figures, a regression analysis models the single-session relationship between stride length (the VOI) and speed (the predictor). However, it should be noted that other relationships may be modeled, including relationships between any combination of one of the VOIs and one of the predictors described in this detailed description.

In embodiments, the general linear model of the relationship between the VOI and the predictor (P) is modeled by a multiple regression represented equation as follows:

VOI˜β₀+β₁*P+β₂*P+ϵ

Equation 9—Regression Relationship Between VOI and Predictor

In Equation 9 above, β₀ represents an intercept, i.e., an expected value of the VOI (e.g., stride length) at a fixed value of the predictor (e.g., fixed speed value) for session-to-session comparison. In preferred embodiments, the fixed value of the predictor is a common value of the predictor across data sets corresponding to two or more sessions (e.g., a speed value that is achieved in each of the two or more sessions, or at least in most sessions). The intercept may, for example, relate the fixed value of the predictor to an average value of the VOI at the fixed value of the predictor (e.g., average stride length at a fixed speed), so as to effectively “center” the model at the intercept and thereby provide session-to-session transferability of the modeling equation.

Still referring to Equation 9, β₁ represents a primary relation coefficient that relates the VOI (e.g., stride length) to the predictor (e.g., speed). β₂ represents a gradient coefficient, which effectively adjusts the relationship of the VOI to the predictor based on variation of the relationship at different elevations. For example, in the relationship between stride length and speed, a same speed value may correspond to a shorter stride length at an uphill elevation gradient, and may correspond to a longer stride length at a downhill elevation gradient. β₃ represents a within-session time coefficient, which effectively adjusts the relationship of the VOI to the predictor based upon a time marker within a session relative to the intercept (e.g., when the relationship varies based upon whether the runner is “warming up” at the beginning of a session, fatiguing at the end of a running session, etc.). In various embodiments, additional, fewer, and/or alternative coefficients may be present, e.g., when the relationship between the VOI and the predictor varies as a function of one or more other variables (e.g., heart rate, step frequency, blood oxygen saturation, humidity in the environment, weight of the runner, footwear used by the runner, running surface used by the runner, etc.).

In any case, still referring to Equation 9, a residual mean absolute error (MAE) c provides a measure of the variance of the VOI (e.g., variance in the VOI in two or more instances under identical values of the aforementioned predictor and coefficients). Although variability in the VOI may be related to changes in health or performance, analyzing these changes particularly in uncontrolled settings (e.g., outside of a laboratory) is difficult. Accordingly, the error c seeks to account for deviations in the VOI that is unexplained by the aforementioned predictor and coefficient(s) in the modeling equation, without needing to explicitly define or model the source(s) of the deviations. In some embodiments, variability in the VOI may be determined or expressed in another manner, e.g., based upon residual plots for the VOI, based upon binning observations of the VOI within certain ranges of the predictors, and/or still other data processing techniques.

In various embodiments, additional or alternative measures and/or interactions may be added or substituted into the model described herein. In some embodiments, the MAE may be transformed to a coefficient of variation, expressed as a percent of the centered intercept measure (i.e., a percent of the VOI at the intercept). Moreover, if the modeled relationship between the predictor and the VOI proved to be nonlinear, the predictor in the modeling equation may be transformed (e.g., log-transformed) or given a polynomial term (e.g., in Equation 9 above, P may be substituted with P², P³, the square or cubic root of P, etc.). Moreover, because the approach described herein uses a statistical framework, the measures described herein may be tested within the framework for their significance, i.e., the probability that a given measure exhibits a meaningful effect on the relationship in each of the one or more running sessions being analyzed (and thus whether the measure should be represented in the modeling equation).

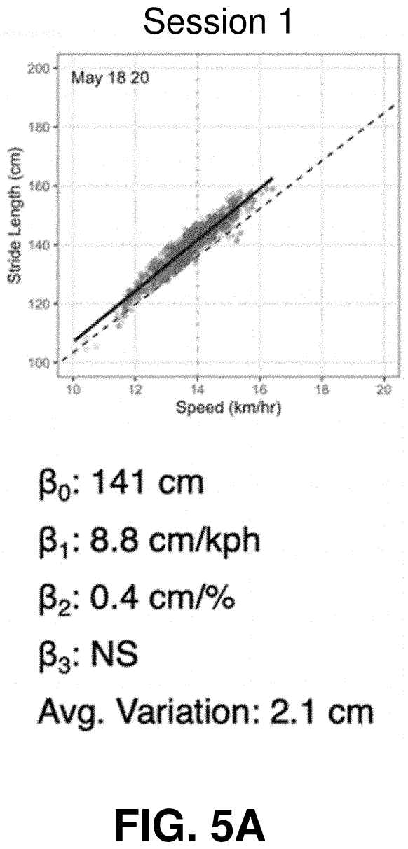

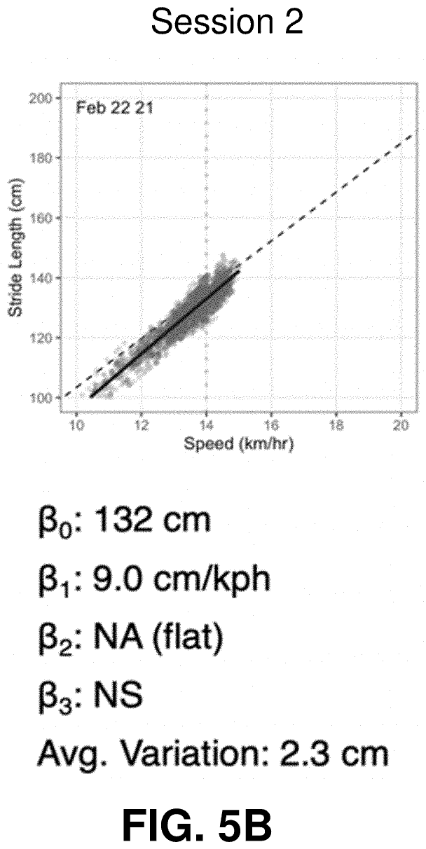

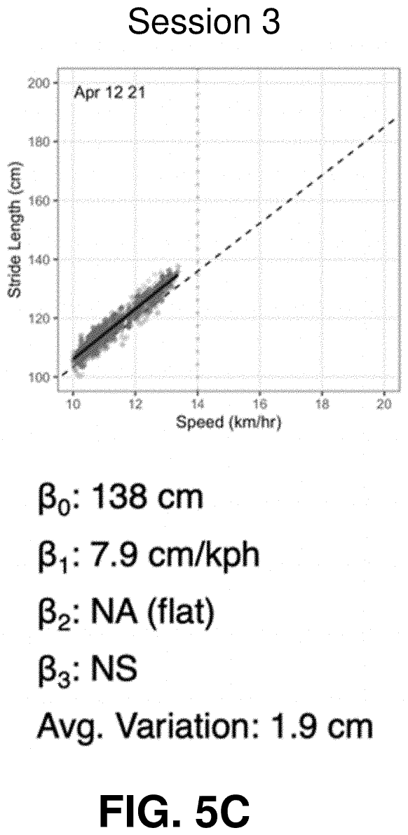

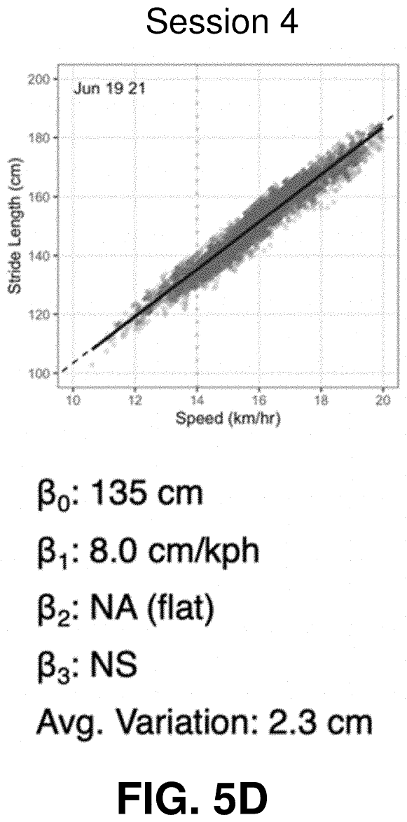

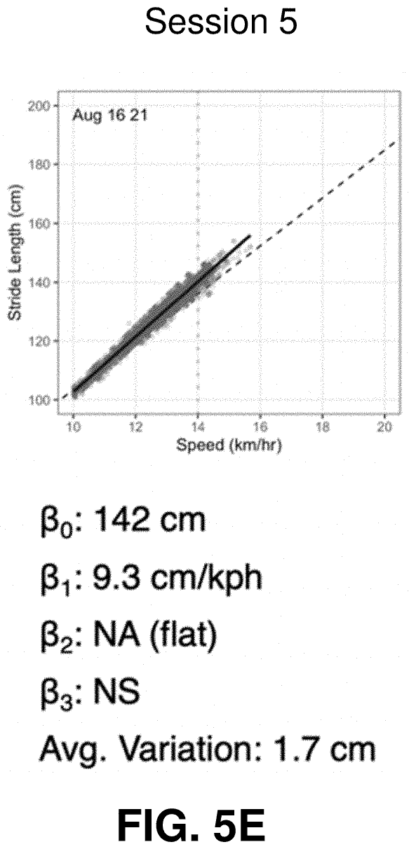

In accordance with the techniques described herein, FIGS. 5A-5E respectively illustrate single-session plots of a VOI (stride length) and a predictor (speed) during each of five running sessions (Sessions 1, 2, 3, 4, and 5) during which data was collected from a runner. In each plot, a diagonal dashed line indicates the line of the modeling equation from the respective running session. Each point (dot) on the graph represents a sample collected from a GPS watch and process/transformed using the techniques described in the foregoing sections of this detailed description. As can be observed from FIGS. 5A-5E, beta coefficients and the residual mean absolute error vary between the modeling equations corresponding to each of the respective running sessions.

More particular, first referring to FIG. 5A, Session 1 corresponds to the observed runner's return to training after a long layoff period. Compared to most of the rest of the sessions, a longer stride length was observed across speeds, as indicated by a higher intercept coefficient β₀ (the vertical dashed line centered at 14 km/hr). Now referring to FIG. 5B, Session 2 was the observed runner's last running session before being diagnosed with a tibial stress fracture. In Session 2, a shorter stride length was observed across speeds.

Next referring to FIG. 5C, Session 3 was the runner's first running session after rehabilitation from the tibial stress fracture. In Session 3, a higher stride length was observed across speeds, with lower variability in stride length relative to Sessions 1 and 2. In FIG. 5D, Session 4 was a running session after several months of consistent training by the runner upon rehabilitation from the stress fracture, and was generally characterized by an overall return to normal metrics for the runner. Finally, referring to FIG. 5E, Session 5 was a session during a period when the runner ran several good race performances, indicating advanced fitness. From Session 5 was observed an increased stride length across speeds, an increased speed relationship to stride length (β₁), and lower variability as compared to Sessions 1, 2, 3, and 4.

Accordingly, in view of the observations depicted in FIGS. 5A-5E, it might be questioned whether the modeled coefficients may exhibit identifiable trends over more than five sessions or over a longer length of time, and further, whether such coefficient values or trends may correlate to or be predictive of improved performance, risk of injury, etc.

### Multiple Session Model Aggregation

A sixth stage of a longitudinal analysis may include running the single-session model (defined at the previous stage) on data from each of another one, two, three, or more running sessions. By modeling the beta coefficients of the GLM modeling equation over time, values or trends in the coefficients may be analyzed to determine whether the values and/or trends may explain, asses, and/or predict changes in runner performance (e.g., improved speed or energy economy) and/or changes in runner health (e.g., reduced risk of acute or stress-induced injury).

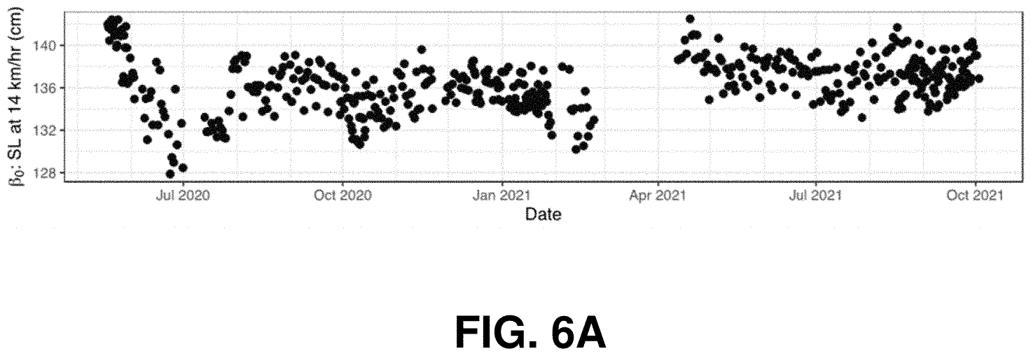

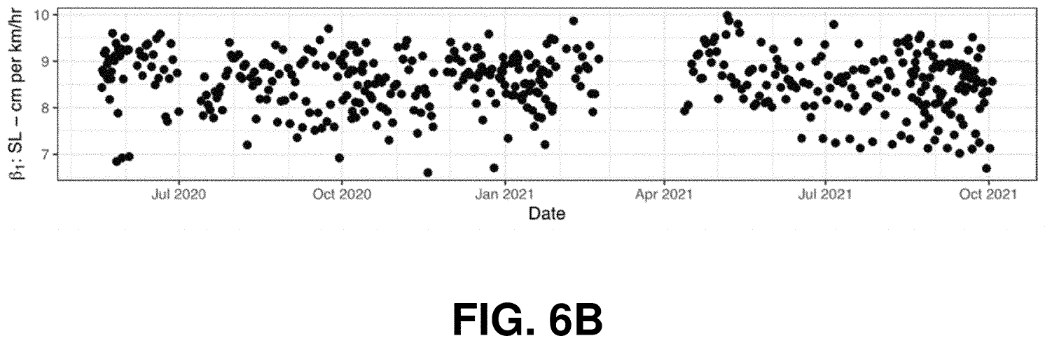

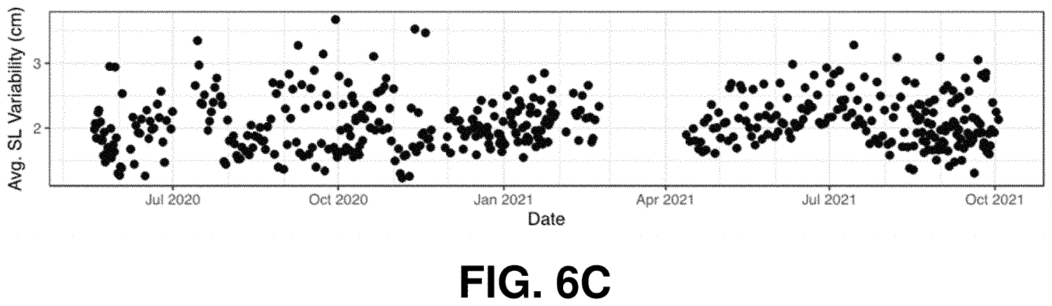

In accordance with these techniques, FIGS. 6A-6C respectively plot values of beta coefficients β₀, β₁, and β₂ for the runner across running sessions over a period of approximately eighteen months. Each plot is informed by over 2.2 million data samples across 471 running sessions, with each point representing the value of the given beta coefficient for a single session.

In FIG. 6A, a drop in stride length across speeds (AO can be observed from the running sessions in May 2020 through July 2020, as the runner returned to training after a long injury layoff. The runner experienced another brief period of injury during July 2020. Another drop in stride length was observed as the runner incurred a stress fracture in February 2021, which was followed by a drastic change in stride length after return to training. In FIG. 6B, a general increase in the stride length to speed relationship coefficient β₁ was observed immediately preceding and following the stress fracture, and a decrease in β₁ was observed through May-October 2021 as the runner regained fitness and performance capacity. Moving to FIG. 6C, increase in stride length variability (e) was observed as the runner increased training volume and concomitant fatigue from April-July 2021. A decrease in stride length variability was observed after the runner's adaptation of training and general elevation of performance capacity.

### Extensions of Longitudinal Analysis Techniques

Still additional extensions of the techniques described herein may be envisioned, in various embodiments.

In some embodiments, the model approach described herein may include additional predictor terms to examine their effects on the VOI. For example, a coefficient corresponding to footwear and/or running surface may be used to examine the effects of different shoes and/or surfaces on the relationship between the primary predictor and VOI (e.g., stride length and speed) within a session and across a plurality of sessions. As another example, the model may additional examine the effect of an environmental condition (e.g., temperature or humidity) on the relationship between the primary predictor and VOI (e.g., stride length and speed) within and/or across sessions. It should be noted that, using the general linear model (GLM), the additional coefficient(s) can be discrete variables (e.g., different shoe models) or continuous coefficients (e.g., temperature or humidity). Any of these variables may be assessed to determine whether they have a statistically significant influence of the VOI and/or the relationships between the primary predictor(s) and VOI.

In some embodiments, the longitudinal profile described in the foregoing sections may be recursively updated to serve as a base dataset against which to test new data. That is, the new data may be analyzed with similar techniques to determine whether the new data exhibits different values and/or trends as compared to previous data, with these analyses being specific to the particular runner. For example, following the example provided with respect to the preceding figures, it may be determined whether a stride length vs. speed relationship in the new data is significantly different than the same relationship modeled by previous sessions. In a more particular example, a current session's relationship may be tested against the relationship of running sessions over the previous seven, fourteen, or thirty days, where the current session is assigned an additional model coefficient and tested for significant effect within the larger data set including the previous seven, fourteen, or thirty days. Means of selecting the data against which to test the current session could include, but isn't limited to, expanding/shrinking the time window of the previous sessions, introducing a time lag, using an exponential decay for the significance of the oldest sessions, etc.

Still additionally or alternatively, in some embodiments, the analyses described herein may be expanded to include multiple VOIs from the same dataset (e.g., stride length and heart rate). Multiple regression approaches within the GLM framework and/or alternative methods (e.g., statistical or machine learning approaches) may examine not only the relationships and changes of the VOIs to the predictor(s) and the changes therein, but also how those relationships interact to change the VOIs together. For example, it may be examined how the relationships between stride length and heart rate change with speed change over time. Changes in such relationships may indicate some adaptations by the runner and/or deteriorations in the runner's performance and/or health, which may not ordinarily be revealed by analysis of a single VOI.

Still yet additionally or alternatively, in some embodiments, the techniques described herein may be utilized to form a profile for a runner that may be used as a means of determination or verification of identify. For example, if an established coefficient of a relationship or a trend thereof exhibits a quality distinct enough to uniquely identify a runner from among two or more runners, new data may be related to the previously modeled data to determine or verify that the new data corresponds to the same runner. New data may be related to the runner, for example, by determining that a coefficient of a relationship between a VOI and predictor falls within a range of values around an average coefficient particular to the runner.

In some embodiments, the longitudinal analysis techniques described herein may be extended to other forms of human exercise. For example, the techniques herein may be extended to cycling, whereby a relationship may be established between one or more VOIs (e.g., heart rate, blood pressure, speed, force exerted on a pedal, etc.), and one or more predictors (e.g., speed, heart rate, etc.).

It should be noted that the techniques described herein may still otherwise be modulated or extended, in various embodiments. Particularly, various VOIs may be modeled with respect to various predictors, with various measurement devices and data collection/aggregation strategies. For example, with an additional accelerometer as a measuring device, additional mechanical VOIs such as spring-mass system parameters of the runner may be evaluated via direct measure or via calculation/transformation from other collected metrics. As another example, using a continuous glucose monitor as a measuring device, blood glucose content may be modeled as a VOI within or across sessions relative to a predictor. As still another example, using a digestible internal temperature sensor as a measuring device, the runner's core temperature may be modeled as the VOI relative to the predictor.

Moreover, while the example approach described in the foregoing sections used a GLM framework, additional or alternate techniques or approaches may be used to test the significance (or probability thereof) of any given predictor upon a VOI. Other statistical approaches to these techniques may include Bayesian approaches, econometric approaches, time series analyses or time-varying effect models, machine learning approaches, and/or still other approaches.

### Example Computer-Implemented Method

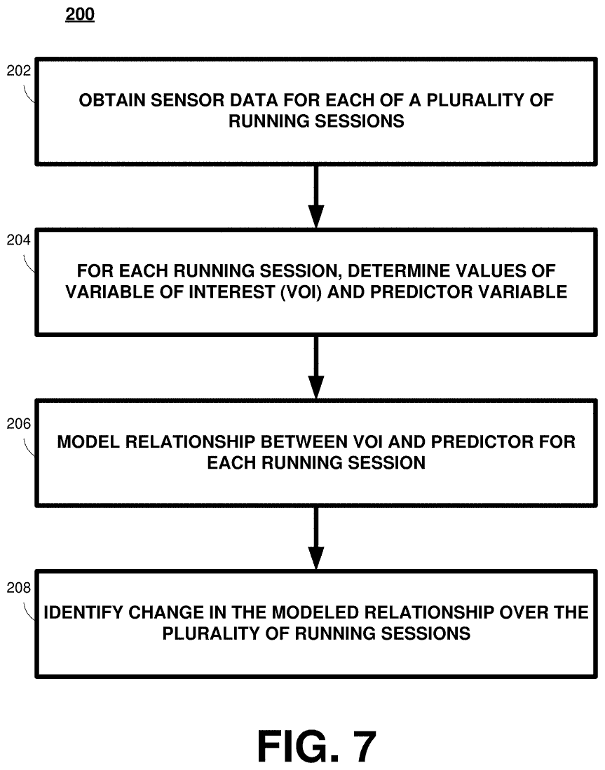

FIG. 7 illustrates a block diagram of an example computer-implemented method 200 associated with longitudinal analysis of human running gait metrics. The method 200 may be performed, for example, via one or more of the computing devices with respect to FIG. 1 (e.g., via the one or more servers 52 and/or another one or more computing devices configured to perform the actions described herein.

The method 200 includes obtaining, via one or more sensing devices, for each of a plurality of running session of a human runner, sensor data indicative of the respective running session (202). The one or more sensing devices may include a smart watch, a hip-mounted sensor, an inertial measurement unit, a force plate, and/or any other suitable sensing devices described herein.

The method 200 further includes, for each respective running session, determining, based upon the sensor data, values of a variable of interest (VOI, e.g., stride length, heart rate, stiffness, contact time, touchdown angle, etc.) and a predictor variable (e.g., speed for a respective plurality of data points over the respective running session (204). That is, VOIs and/or predictors may be obtained either by being directly sensed by one or more sensing devices, or being mathematically determined based upon the measurements of the sensing devices.

The method 200 still further includes, for each respective running session, modeling a statistical relationship between the VOI and the predictor variable (206). The model may include a general linear model (e.g., a multiple regression model), in some embodiments, and may particularly include coefficients modeling any of (1) an intercept value of the VOI, (2) a gradient coefficient relating a variation of the modeled relationship based upon elevation, and/or (3) a time coefficient relating a variation of the modeled relationship based upon a time within a running session (e.g., warming up, winding down, etc.).

The method 200 still yet further includes, based upon the modeled relationship for each respective running session, identifying a change in the statistical relationship between the VOI and predictor variable over the plurality of running sessions (208). Identifying the change may be used, for example, to identify an improvement or deterioration in performance by the runner, or a risk of injury to the runner based upon a trend in the statistical relationship over the plurality of running sessions.

The method 200 may include additional, fewer, and/or alternate actions, in various embodiments, including any appropriate action(s) described in this detailed description.

### Additional Considerations

Although the above text sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments could be implemented, using either current technology or technology developed after the filing date of this patent, which would still fall within the scope of the claims. By way of example, and not limitation, the disclosure herein contemplates at least the following aspects:

1. A computer-implemented method performed via one or more processors, the method comprising (1) obtaining, via one or more sensing devices, for each of a plurality of running sessions of a human runner, sensor data indicative of the respective running session, (2) for each respective running session, determining, based upon the sensor data, values of a variable of interest (VOI) and a predictor variable for a respective plurality of data points over the respective running session, (3) for each respective running session, modeling a statistical relationship between at least the VOI and the predictor variable, and (4) based upon the modeled relationship for each respective running session, identifying a change in the statistical relationship between the VOI and predictor variable over the plurality of running sessions.

2. The computer-implemented method of aspect 1, wherein the one or more sensing devices include a smart watch

3. The computer-implemented method of aspect 1 or 2, wherein the one or more sensing devices include an accelerometer or gyroscope.

4. The computer-implemented method of any of aspects 1-3, wherein the VOI is a stride length of the runner.

5. The computer-implemented method of any of aspects 1-3, wherein the VOI is a heart rate of the runner.

6. The computer-implemented method of any of aspects 1-3, wherein the VOI includes a spring-mass parameter of the runner.

7. The computer-implemented method of any of aspects 1-3, wherein the VOI is a running speed of the runner.

8. The computer-implemented method of any of aspects 1-6, wherein the predictor variable is a running speed of the runner.

9. The computer-implemented method of any of aspects 1-6 or 8, wherein the predictor variable is one of a stride length, a heart rate, or a spring-mass parameter of the runner.

10. The computer-implemented method of any of aspects 1-9, wherein modeling the statistical relationship includes generating a general linear model of the relationship between the VOI and the predictor.

11. The computer-implemented method of any of aspects 1-10, wherein modeling the statistical relationship includes determining a gradient coefficient relating the predictor to the VOI.

12. The computer-implemented method of any of aspects 1-11, wherein modeling the statistical relationship includes determining a time coefficient relating the predictor to the VOI.

13. The computer-implemented method of any of aspects 1-12, wherein modeling the0 statistical relationship includes determining a footwear coefficient relating the predictor to the VOI.

14. The computer-implemented method of any of aspects 1-13, wherein modeling the statistical relationship includes determining a running surface coefficient relating the predictor to the VOI.

15. The computer-implemented method of any of aspects 1-14, wherein modeling the statistical relationship includes determining a runner weight coefficient relating the predictor to the VOI.

16. The computer-implemented method of any of aspects 1-15, further comprising identifying an improvement to performance of the runner based upon the change in the statistical relationship over at least a portion of the plurality of running sessions.

17. The computer-implemented method of any of aspects 1-16, further comprising identifying an injury risk or probability of injury experienced by the runner of the runner based upon the change in the statistical relationship over at least a portion of the plurality of running sessions.

18. The computer-implemented method of any of aspects 1-17, further comprising (1) generating, based upon the modeled relationship for each respective running session, a profile of the relationship between the VOI and predictor, the profile being specific to the runner, (2) obtaining values of the VOI and predictor for an additional running session; (3) modeling the relationship between the VOI and the predictor for the additional running session; and (4) comparing the model for the additional running session to the model for the plurality of running sessions to determine that the model for the additional running session falls within a range defined by the model of the plurality of running sessions.

19. The computer-implemented method of any of aspects 1-17, further comprising: (1) generating, based upon the modeled relationship for each respective running session, a profile of the relationship between the VOI and predictor, the profile being specific to the runner, (2) obtaining values of the VOI and predictor for an additional running session, (3) modeling the relationship between the VOI and the predictor for the additional running session; and (4) comparing the model for the additional running session to the model for the plurality of running sessions to determine an improvement or deterioration in performance by the runner during the additional running session compared to the plurality of running sessions.

20. The computer-implemented method of any of aspects 1-17, further comprising (1) generating, based upon the modeled relationship for each respective running session, a profile of the relationship between the VOI and predictor, the profile being specific to the runner; (2) obtaining values of the VOI and predictor for an additional running session; (3) modeling the relationship between the VOI and the predictor for the additional running session; and (4) comparing the model for the additional running session to the model for the plurality of running sessions to determine a change in health status or a risk of injury to the runner in the additional running session.

21. The computer-implemented method of any of aspects 1-20, wherein the statistical relationship relates the VOI and one or more additional VOIs to the predictor.

22. The computer-implemented method of any of aspects 1-20, wherein the statistical relationship relates the VOI to the predictor variable and one or more additional predictor variables.

23. The computer-implemented method of any of aspects 1-20, wherein the statistical relationship relates the VOI and one or more additional VOIs to the predictor variable and one or more additional predictor variables.

24. The computer-implemented method of any one of aspects 1-23, in combination with any other suitable one of aspects 1-23.

25. A computing system comprising one or more processors and one or more computer memories storing non-transitory, computer executable instructions that, when executed via the one or more processors, cause the computing system to (1) obtain, via one or more sensing devices, for each of a plurality of running sessions of a human runner, sensor data indicative of the respective running session, (2) for each respective running session, determine, based upon the sensor data, values of a variable of interest (VOI) and a predictor variable for a respective plurality of data points over the respective running session, (3) for each respective running session, model a statistical relationship between at least the VOI and the predictor variable; and (4) based upon the modeled relationship for each respective running session, identify a change in the statistical relationship between the VOI and predictor variable over the plurality of running sessions.

26. The computing system of aspect 25, wherein the one or more sensing devices include a smart watch.

27. The computing system of aspect 25 or 26, wherein the one or more sensing devices include an accelerometer or gyroscope.

28. The computing system of any of aspects 25-27, wherein the VOI is a stride length of the runner.

29. The computing system of any of aspects 25-27, wherein the VOI is a heart rate of the runner.

30. The computing system of any of aspects 25-27, wherein the VOI includes a spring-mass parameter of the runner.

31. The computing system of any of aspects 25-27, wherein the VOI is a running speed of the runner.

32. The computing system of any of aspects 25-30, wherein the predictor variable is a running speed of the runner.

33. The computing system of any of aspects 25-30 or 32, wherein the predictor variable is one of a stride length, a heart rate, or a spring-mass parameter of the runner.

34. The computing system of any of aspects 25-33, wherein the instructions to model the statistical relationship include instructions to generate a general linear model of the relationship between the VOI and the predictor.

35. The computing system of any of aspects 25-34, wherein the instructions to model the statistical relationship include instructions to determine a gradient coefficient relating the predictor to the VOI.

36. The computing system of any of aspects 25-35, wherein the instructions to model the statistical relationship include instructions to determine a time coefficient relating the predictor to the VOI.

37. The computing system of any of aspects 25-36, wherein the instructions to model the statistical relationship include instructions to determine a footwear coefficient relating the predictor to the VOI.

38. The computing system of any of aspects 25-37, wherein the instructions to model the statistical relationship include instructions to determine a running surface coefficient relating the predictor to the VOI.

39. The computing system of any of aspects 25-38, wherein the instructions to model the statistical relationship include instructions to determine a runner weight coefficient relating the predictor to the VOI.

40. The computing system of any of aspects 25-39, wherein the non-transitory, computer executable instructions, when executed via the one or more processors, further cause the computing system to identify an improvement to performance of the runner based upon the change in the statistical relationship over at least a portion of the plurality of running sessions.

41. The computing system of any of aspects 25-40, wherein the non-transitory, computer executable instructions, when executed via the one or more processors, further cause the computing system to identify an injury risk or probability of injury experienced by the runner of the runner based upon the change in the statistical relationship over at least a portion of the plurality of running sessions.

42. The computing system of any of aspects 25-41, wherein the non-transitory, computer executable instructions, when executed via the one or more processors, further cause the computing system to (1) generate, based upon the modeled relationship for each respective running session, a profile of the relationship between the VOI and predictor, the profile being specific to the runner, (2) obtain values of the VOI and predictor for an additional running session, (3) model the relationship between the VOI and the predictor for the additional running session, and (4) compare the model for the additional running session to the model for the plurality of running sessions to determine that the model for the additional running session falls within a range defined by the model of the plurality of running sessions.

43. The computing system of any of aspects 25-41, wherein the non-transitory, computer executable instructions that, when executed via the one or more processors, further cause the computing system to (1) generate, based upon the modeled relationship for each respective running session, a profile of the relationship between the VOI and predictor, the profile being specific to the runner, (2) obtain values of the VOI and predictor for an additional running session, (3) model the relationship between the VOI and the predictor for the additional running session, and (4) compare the model for the additional running session to the model for the plurality of running sessions to determine an improvement or deterioration in performance by the runner during the additional running session compared to the plurality of running sessions.

44. The computing system of any of aspects 25-41, wherein the non-transitory, computer executable instructions that, when executed via the one or more processors, further cause the computing system to (1) generate, based upon the modeled relationship for each respective running session, a profile of the relationship between the VOI and predictor, the profile being specific to the runner, (2) obtain values of the VOI and predictor for an additional running session, (3) model the relationship between the VOI and the predictor for the additional running session; and (4) compare the model for the additional running session to the model for the plurality of running sessions to determine a change in health status or a risk of injury to the runner in the additional running session.

45. The computing system of any of aspects 25-44, wherein the statistical relationship relates the VOI and one or more additional VOIs to the predictor.

46. The computing system of any of aspects 25-44, wherein the statistical relationship relates the VOI to the predictor variable and one or more additional predictor variables.

47. The computing system of any of aspects 25-44, wherein the statistical relationship relates the VOI and one or more additional VOIs to the predictor variable and one or more additional predictor variables.

48. The computing system of any one of aspects 25-47, configured to perform the actions of any other suitable one of aspects 25-47.

49. The computing system of any of one aspects 25-48, configured to perform the method of any one of aspects 1-24.

50. One or more computer readable media storing non-transitory, computer executable instructions that, when executed via one or more processors of one or more computers, cause the one or more computers to (1) obtain, via one or more sensing devices, for each of a plurality of running sessions of a human runner, sensor data indicative of the respective running session, (2) for each respective running session, determine, based upon the sensor data, values of a variable of interest (VOI) and a predictor variable for a respective plurality of data points over the respective running session, (3) for each respective running session, model a statistical relationship between at least the VOI and the predictor variable, and (4) based upon the modeled relationship for each respective running session, identify a change in the statistical relationship between the VOI and predictor variable over the plurality of running sessions.

51. The one or more computer readable media of aspect 50, wherein the one or more sensing devices include a smart watch.

52. The one or more computer readable media of aspect 50 or 51, wherein the one or more sensing devices include an accelerometer or gyroscope.

53. The one or more computer readable media of any of aspects 50-52, wherein the VOI is a stride length of the runner.

54. The one or more computer readable media of any of aspects 50-52, wherein the VOI is a heart rate of the runner.

55. The one or more computer readable media of any of aspects 50-52, wherein the VOI includes a spring-mass parameter of the runner.

56. The one or more computer readable media of any of aspects 50-52, wherein the predictor variable is a running speed of the runner.

57. The one or more computer readable media of any of aspects 50-55, wherein the predictor variable is a running speed of the runner.

58. The one or more computer readable media of any of aspects 50-55 or 57, wherein the predictor variable is one of a stride length, a heart rate, or a spring-mass parameter of the runner.

59. The one or more computer readable media of any of aspects 50-58, wherein the instructions to model the statistical relationship include instructions to generate a general linear model of the relationship between the VOI and the predictor.

60. The one or more computer readable media of any of aspects 50-59, wherein the instructions to model the statistical relationship include instructions to determine a gradient coefficient relating the predictor to the VOI.

61. The one or more computer readable media of any of aspects 50-60, wherein the instructions to model the statistical relationship include instructions to determine a time coefficient relating the predictor to the VOI.

62. The one or more computer readable media of any of aspects 50-61, wherein the instructions to model the statistical relationship include instructions to determine a footwear coefficient relating the predictor to the VOI.

63. The one or more computer readable media of any of aspects 50-62, wherein the instructions to model the statistical relationship include instructions to determine a running surface coefficient relating the predictor to the VOI.

64. The one or more computer readable media of any of aspects 50-63, wherein the instructions to model the statistical relationship include instructions to determine a runner weight coefficient relating the predictor to the VOI.

65. The one or more computer readable media of any of aspects 50-64, wherein the non-transitory, computer executable instructions that, when executed via the one or more processors, further cause the one or more computers to identify an improvement to performance of the runner based upon the change in the statistical relationship over at least a portion of the plurality of running sessions.

66. The one or more computer readable media of any of aspects 50-65, wherein the non-transitory, computer executable instructions that, when executed via the one or more processors, further cause the one or more computers to identify an injury risk or probability of injury experienced by the runner of the runner based upon the change in the statistical relationship over at least a portion of the plurality of running sessions.

67. The one or more computer readable media of any of aspects 50-66, wherein the non-transitory, computer executable instructions that, when executed via the one or more processors, further cause the one or more computers to (1) generate, based upon the modeled relationship for each respective running session, a profile of the relationship between the VOI and predictor, the profile being specific to the runner, (2) obtain values of the VOI and predictor for an additional running session, (3) model the relationship between the VOI and the predictor for the additional running session, and (4) compare the model for the additional running session to the model for the plurality of running sessions to determine that the model for the additional running session falls within a range defined by the model of the plurality of running sessions.

68. The one or more computer readable media of any of aspects 50-66, wherein the non-transitory, computer executable instructions that, when executed via the one or more processors, further cause the one or more computers to (1) generate, based upon the modeled relationship for each respective running session, a profile of the relationship between the VOI and predictor, the profile being specific to the runner, (2) obtain values of the VOI and predictor for an additional running session, (3) model the relationship between the VOI and the predictor for the additional running session, and (4) compare the model for the additional running session to the model for the plurality of running sessions to determine an improvement or deterioration in performance by the runner during the additional running session compared to the plurality of running sessions.

69. The one or more computer readable media of any of aspects 50-66, wherein the non-transitory, computer executable instructions that, when executed via the one or more processors, further cause the one or more computers to (1) generate, based upon the modeled relationship for each respective running session, a profile of the relationship between the VOI and predictor, the profile being specific to the runner, (2) obtain values of the VOI and predictor for an additional running session, (3) model the relationship between the VOI and the predictor for the additional running session, and (4) compare the model for the additional running session to the model for the plurality of running sessions to determine a change in health status or a risk of injury to the runner in the additional running session.

70. The one or more computer readable media of any of aspects 50-69, wherein the statistical relationship relates the VOI and one or more additional VOIs to the predictor.

71. The one or more computer readable media of any of aspects 50-69, wherein the statistical relationship relates the VOI to the predictor variable and one or more additional predictor variables.

72. The one or more computer readable media of any of aspects 50-69, wherein the statistical relationship relates the VOI and one or more additional VOIs to the predictor variable and one or more additional predictor variables.

73. The one or more computer readable media of any one of aspects 50-72, configured to cause the one or more computers to perform the actions of any other suitable one of aspects 50-72.

74. The one or more computer readable media of any one of aspects 50-72, configured to cause a computing system to perform the actions of any one of aspects 25-49.

75. The one or more computer readable media of any one of aspects 50-72, configured to perform the method of any one of aspects 1-24.

76. Any one of aspects 1-75 in combination with any other suitable one of aspects 1-75.

Thus, many modifications and modifications and variations may be made in the techniques and structures described and illustrated herein without departing from the spirit and scope of the present claims. Accordingly, it should be understood that the methods and apparatus described herein are illustrative only and are not limiting upon the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a non-transitory, machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that may be permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that may be temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules may provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it may be communicatively coupled. A further hardware module may then, at a later time, access the memory device to retrieve and process the stored output. Hardware modules may also initiate communications with input or output devices, and may operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment, or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment may be included in at least one embodiment. The appearances of the phrase “in one embodiment” in various places in the specification are not necessarily all referring to the same embodiment.

As used herein, the terms “comprises,” “comprising,” “may include,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also may include the plural unless it is obvious that it is meant otherwise.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. § 112, sixth paragraph.

What is claimed is:

1. A computer-implemented method performed via one or more processors, the method comprising:
obtaining, via one or more sensing devices, for each of a plurality of running sessions of a human runner, sensor data indicative of the respective running session;
for each respective running session, determining, based upon the sensor data, values of a variable of interest (VOI) and a predictor variable for a respective plurality of data points over the respective running session;
for each respective running session, modeling a statistical relationship between at least the VOI and the predictor variable; and
based upon the modeled relationship for each respective running session, identifying a change in the statistical relationship between the VOI and predictor variable over the plurality of running sessions.

2. The computer-implemented method of claim 1, wherein the one or more sensing devices include a smart watch.

3. The computer-implemented method of claim 1, wherein the one or more sensing devices include an accelerometer or gyroscope.

4. The computer-implemented method of claim 1, wherein the VOI is one of a stride length of the runner, a heart rate of the runner, a spring-mass parameter of the runner, or a running speed of the runner.

5. The computer-implemented method of claim 1, wherein the predictor variable is one of a running speed of the runner, a stride length of the runner, a heart rate of the runner, or a spring-mass parameter of the runner.

6. The computer-implemented method of claim 1, wherein modeling the statistical relationship includes generating a general linear model of the relationship between the VOI and the predictor.

7. The computer-implemented method of claim 1, wherein modeling the statistical relationship includes determining at least one of a gradient coefficient, a time coefficient, a footwear coefficient, a running surface coefficient, or a runner weight coefficient relating the predictor to the VOI.

8. The computer-implemented method of claim 1, further comprising identifying an improvement to performance of the runner based upon the change in the statistical relationship over at least a portion of the plurality of running sessions.

9. The computer-implemented method of claim 1, further comprising identifying an injury risk or probability of injury experienced by the runner of the runner based upon the change in the statistical relationship over at least a portion of the plurality of running sessions.

10. The computer-implemented method of claim 1, further comprising:
generating, based upon the modeled relationship for each respective running session, a profile of the relationship between the VOI and predictor, the profile being specific to the runner;
obtaining values of the VOI and predictor for an additional running session;
modeling the relationship between the VOI and the predictor for the additional running session; and
comparing the model for the additional running session to the model for the plurality of running sessions to determine at least one of (1) a change in health status or a risk of injury to the runner in the additional running session, (2) an improvement or deterioration in performance by the runner during the additional running session compared to the plurality of running sessions, or (3) that the model for the additional running session falls within a range defined by the model of the plurality of running sessions.

11. A computing system comprising:
one or more processors; and
one or more computer memories storing non-transitory, computer executable instructions that, when executed via the one or more processors, cause the computing system to:
obtain, via one or more sensing devices, for each of a plurality of running sessions of a human runner, sensor data indicative of the respective running session;
for each respective running session, determine, based upon the sensor data, values of a variable of interest (VOI) and a predictor variable for a respective plurality of data points over the respective running session;
for each respective running session, model a statistical relationship between at least the VOI and the predictor variable; and
based upon the modeled relationship for each respective running session, identify a change in the statistical relationship between the VOI and predictor variable over the plurality of running sessions.

12. The computing system of claim 11, wherein the one or more sensing devices include a smart watch.

13. The computing system of claim 11, wherein the one or more sensing devices include an accelerometer or gyroscope.

14. The computing system of claim 11, wherein the VOI is one of a stride length of the runner, a heart rate of the runner, a spring-mass parameter of the runner, or a running speed of the runner.

15. The computing system of claim 11, wherein the predictor variable is one of a running speed of the runner, a stride length of the runner, a heart rate of the runner, or a spring-mass parameter of the runner.

16. The computing system of claim 11, wherein the instructions to model the statistical relationship include instructions to generate a general linear model of the relationship between the VOI and the predictor.

17. The computing system 11, wherein the instructions to model the statistical relationship include instructions to determine at least one of a gradient coefficient, a time coefficient, a footwear coefficient, a running surface coefficient, or a runner weight coefficient relating the predictor to the VOI.

18. The computing system of claim 11, wherein the non-transitory, computer executable instructions, when executed via the one or more processors, further cause the computing system to identify, based upon the change in the statistical relationship over at least a portion of the plurality of running sessions, at least one of (1) an improvement to performance of the runner or (2) an injury risk or probability of injury experienced by the runner of the runner.

19. The computing system of claim 11, wherein the non-transitory, computer executable instructions, when executed via the one or more processors, further cause the computing system to:
generate, based upon the modeled relationship for each respective running session, a profile of the relationship between the VOI and predictor, the profile being specific to the runner;
obtain values of the VOI and predictor for an additional running session;
model the relationship between the VOI and the predictor for the additional running session; and
compare the model for the additional running session to the model for the plurality of running sessions to determine at least one of (1) a change in health status or a risk of injury to the runner in the additional running session, (2) an improvement or deterioration in performance by the runner during the additional running session compared to the plurality of running sessions, or (3) that the model for the additional running session falls within a range defined by the model of the plurality of running sessions.

20. One or more computer readable media storing non-transitory, computer executable instructions that, when executed via one or more processors of one or more computers, cause the one or more computers to:
obtain, via one or more sensing devices, for each of a plurality of running sessions of a human runner, sensor data indicative of the respective running session;
for each respective running session, determine, based upon the sensor data, values of a variable of interest (VOI) and a predictor variable for a respective plurality of data points over the respective running session;
for each respective running session, model a statistical relationship between at least the VOI and the predictor variable; and
based upon the modeled relationship for each respective running session, identify a change in the statistical relationship between the VOI and predictor variable over the plurality of running sessions.
